(19) United States
(12) Patent Application Publication
(10) Pub. No.: US 2023/0226015 A1
(43) Pub. Date: Jul. 20, 2023

(54) DEFINED DOSE CANNABIS PUCK

(71) Applicant: Emerald Health Therapeutics Canada Inc., Vancouver (CA)

(72) Inventors: Christopher Wagner, Vancouver (CA); Nancy Harrison, Vancouver (CA); Freydoun Garabagi, Vancouver (CA)

(21) Appl. No.: 18/179,405

(22) Filed: Mar. 7, 2023

Related U.S. Application Data
(62) Division of application No. 16/981,209, filed on Sep. 15, 2020.
(60) Provisional application No. 62/645,049, filed on Mar. 19, 2018.

(51) Int. Cl. A61K 31/352; A61K 9/20; A61K 31/05

(52) U.S. Cl. CPC A61K 31/352; A61K 9/2095; A61K 31/05

(57) ABSTRACT

This invention relates to novel products of compressed Cannabis flower for medicinal and/or recreational use, and for methods of making such products.

### CROSS-REFERENCE

This application claims benefit of U.S. Provisional Application No. 62/645,049, filed on Mar. 19, 2018, which is incorporated herein by reference in its entirety.

### BACKGROUND OF THE INVENTION

One shot of bourbon, one shot of scotch and one 12 oz. beer all have roughly the same amount of alcohol in them. Users recognize that the mildly intoxicating effects of such drinks are consistent and predictable, despite the wide variety of brands, qualities and prices.

No such equivalency exists for Cannabis. Inspection of a Cannabis plant product by eyesight, taste, or smell provides no reliable guidance as to the amount or potency of physiologically and/or psychotropically active cannabinoids in the product. This presents the user with risks and uncertainty that can only be effectively resolved by consuming small amounts of the sample, waiting for an effect, and then consuming another small part of the sample.

### SUMMARY OF THE INVENTION

The present disclosure relates to compressed Cannabis pucks which contain processed plant source material and which allow the cannabinoid ingredients to be released through vaporization or combustion. The Cannabis pucks provide reliable and consistent defined doses of selected cannabinoids for recreational and/or medicinal users. The Cannabis pucks may mitigate one or more hazards associated with the use of marijuana, particularly concerns regarding consistency of dosing. The present invention provides a family of compressed puck products and methods for preparation thereof

In one embodiment, the invention provides Cannabis pucks comprising a pre-defined total amount of one or more active ingredients in ranges of about 0 mg-100 mg. Active ingredients may be selected from the group including THC, THCA, CBD and CBDA.

In some embodiments, the invention provides Cannabis pucks wherein the active ingredients may be present in predetermined ratios to achieve for example, a desired effect, or for a particular purpose.

In some embodiments, the pucks comprise low THC:THCA ratios. Low THC:THCA pucks may comprise THC in ranges of about 0 mg-1 mg and THCA in ranges of about 9 mg to 90 mg. Low THC:THCA pucks may further comprise CBD in ranges of about 7 mg-75 mg.

In some embodiments, the pucks comprise low THCA:THC ratios. Low THCA:THC pucks may comprise THCA in ranges of about 0 mg-1 mg and THC in ranges of about 9 mg-90 mg. Low THCA:THC pucks may further comprise CBD in ranges of about 7 mg-75 mg.

In some embodiments, the pucks are substantially free of THC-type compounds. Pucks substantially free of THC-type compounds may comprise CBD in ranges of about 7 mg-75 mg, THC in ranges of about 0 mg-1 mg, THCA in ranges of about 0 mg-1 mg.

In some embodiments, the pucks described herein may be for use for direct vaporization, electronic inhalation, ingestion, infusion into edible matrices, or smoking.

In some embodiments, the invention provides methods of producing Cannabis pucks described herein. The methods comprising a) a preparation step; b) a compression step; and c) a recovery step.

Described herein are methods of preparing a Cannabis puck comprising a desired defined dose of one or more desired cannabinoids, the method comprising: (a) preparing or obtaining a ground or milled Cannabis plant material preparation; (b) passing the Cannabis plant material preparation through a mesh or sieve to obtain a retained fraction of the material which is retained in the mesh or sieve and a pass-through fraction of the material which has passed through the mesh or sieve; (c) determining the content of the one or more cannabinoids of interest in the retained fraction and/or the pass-through fraction; (d) based on the content determined in step (c), determining a target amount of retained fraction material, pass-through material, or a combination thereof, to be incorporated into the Cannabis puck such that the puck includes the desired defined dose of the one or more desired cannabinoids; and (e) forming the Cannabis puck from the target amount of retained fraction material, pass-through material, or combination thereof. In some methods described herein, the desired defined dose is selected from the group consisting of: (i) 0.1-100 mg tetrahydrocannabinolic acid (THCA), (ii) 0.1-100 mg tetrahydrocannabidiol (THC), (iii) 0.1-100 mg cannabidiolic acid (CBDA), and/or (iv)1-100 mg cannabidiol acid (CBD). In some methods described herein, the forming comprises a step of compressing the preparation of a) at 300-1500 PSI. In some methods described herein, step a) includes measuring and adjusting each cannabinoid to within +/− 5% of its defined amount. In some methods described herein, the maximum temperature of the preparation during the compressing step is maintained below about 105° C. In some methods described herein, the maximum temperature of the preparation during the compressing step is maintained below about 150° C. In some methods described herein, the compressing step is carried out for a duration of from about 1 second to about 120 seconds. In some methods described herein, the puck comprises: THCA in an amount between 5-165 mg; THC in an amount less than 1.0 mg; and CBDA in an amount between 0.1-70 mg, and wherein the puck has a total mass of 100-500 mg. In some methods described herein, the puck comprises: THCA in an amount less than 5.0 mg, THC in an amount between 1-5 mg or between 5-135 mg; and CBD in an amount between 0.1-70 mg, and wherein the puck has a total mass of 100-500 mg. In some methods described herein, the puck comprises: THCA in an amount less than 1.0 mg, THC in an amount less than 1.0 mg; and CBD in an amount between 5-90 mg, and wherein the puck has a total mass 100-500 mg. In some methods described herein, the Cannabis in the preparation of a) has not been previously exposed to accelerated dehydration at greater than about 100° C. In some methods described herein, following the compressing step, the preparation is heated at a temperature above about 105° C. but below about 150° C. for a duration of about 5 minutes to about 30 minutes. In some methods described herein, prior to step a), the Cannabis preparation has been heated to a temperature above about 105° C. but below about 150° C. for a duration of about 5 minutes to about 30 minutes. In some methods described herein, immediately following the compressing step, the preparation is cooled to a temperature below 10° C. Some methods described herein further comprise after step e), packaging the puck individually in a blister pack impermeable to gas exchange. Some methods described herein further comprise after step e), packaging the puck in a re-sealable multi-puck package impermeable to gas exchange. In some methods described herein, the compressing step is performed in a compression mold shaped to provide a signifier embossed on the puck which provides a visual indication of information on the defined dose of selected cannabinoids in the composition, and/or when the process comprises a further step of laser engraving a signifier on the puck which provides a visual indication of information on the defined dose of selected cannabinoids in the composition. In some methods described herein, the composition comprises packaging for containing the puck, the method further comprising a step of disposing the composition in packaging configured to associate the puck with a signifier which provides visual information on the defined dose of selected cannabinoids in the composition. In some methods described herein, the Cannabis plant material includes material derived from one or more members of a plant variety selected from the group consisting of Cannabis saliva, Cannabis indica, Cannabis riederalis, and hybrids thereof. In some methods described herein, the Cannabis plant variety is Cannabis indica. In some methods described herein, the Cannabis plant material is prepared from Cannabis inflorescence. In some methods described herein, the preparation of step a) further comprises additives selected from among terpenes, terpenoids, puck stabilizers, humectants, vaporization aids, fillers and flavours. In some methods described herein, the Cannabis preparation in a) has been previously ground to sieve through a mesh of not larger than 1.5 mm in any surface dimension. Some methods described herein further comprise: (f) finishing the puck to provide a high gloss surface. In some methods described herein, the ground Cannabis preparation includes about 0.1 to about 1.0 grams total mass. In some methods described herein, the puck possesses a degree of friability such that no more than, or exactly, 1% or 0.66% loss results after a friability test as per USP <1216>. In some methods described herein, the puck possesses a degree of friability that meets specifications provided by USP <1216>. In some methods described herein, the mesh or sieve has a mesh size of 30, 60, or 120. In some methods described herein, the mesh or sieve has an average opening size of about 0.595 mm, about 0.250 mm, or about 0.125 mm. In some methods described herein, the mesh or sieve has a mesh size of 60. In some methods described herein, the mesh or sieve has an average opening size of about 0.250 mm. In some methods described herein, the composition is formed by compressing its components into a predetermined shape. In some methods described herein, the shape is a puck shape. In some methods described herein, the shape is predetermined to be received by a Cannabis vaporizer.

Described herein are compositions comprising a defined dose of: (a) 0.1-100 mg tetrahydrocannabinolic acid (THCA), (b) 0.1-100 mg tetrahydrocannabinol (THC), (c) 0.1-100 mg cannabidiolic acid (CBDA), and/or (d) 0.1-100 mg cannabidiol (CBD) in a friable puck comprising compressed ground Cannabis material. In some compositions described herein, the Cannabis material includes material derived from one or more Cannabis plants from a species selected from the group consisting of Cannabis sativa, Cannabis indica, Cannabis ruderalis, and any hybrid thereof. In some compositions described herein, the Cannabis species is Cannabis indica. In some compositions described herein, the Cannabis material includes material prepared from Cannabis inflorescence. Some compositions described herein further comprise additives selected from the group consisting of terpenes, terpenoids, puck stabilizers, humectants, vaporization aids, fillers flavours, and any combination thereof. In some compositions described herein, the amount of THC is less than a psychotropic dose. In some compositions described herein, the amount of THCA is less than 1.0 mg. Some compositions described herein comprise: THCA in an amount between 5-165 mg; THC in an amount less than 1.0 mg; and CBDA in an amount between 0.1-70 mg, and a total mass 100-500 mg. Some compositions described herein comprise: THCA in an amount less than 5.0 mg; THC in an amount between 1-5 mg or between 5-135 mg; and CBD in an amount between 0.1-70 mg, and a total mass 100-500 mg. Some compositions described herein comprise: THCA in an amount less than 1.0 mg; THC in an amount less than 1.0 mg; and CBD in an amount between 5-90 mg, and a total mass 100-500 mg. In some compositions described herein, the Cannabis material is derived from one or more Cannabis plant varieties selected from the group consisting of Charlottes' Web and one or more other high CBD, low THCA Cannabis plant varieties. Some compositions described herein comprise a plurality of air channels of diameter not greater than 0.5mm and disposed within the friable puck, such that no portion of the puck is greater than about 0.5, 1, 1.5, 2, 2.5, or 3mm from an air surface. Some compositions described herein have a high-gloss surface. Some compositions described herein comprise a blister package, impermeable to gas exchange, for containing the friable puck. In some compositions further described herein, the blister package is configured to contain the friable puck in a sealed, inert gas atmosphere. Some compositions described herein further comprise a re-sealable package for containing one or more of the friable pucks, wherein in a sealed configuration the package provides an environment for containing the pucks that is impermeable to gas exchange. In some compositions described herein, the composition comprises a signifier for providing visual information on one or more defined dose(s) of one or more selected cannabinoids in the composition. In some compositions described herein, the package comprises a signifier for providing visual information on the defined dose(s) of one or more selected cannabinoids in the composition. In some compositions described herein, said puck has a total mass of about 45 mg or from about 0.05 g to about 1.0 g. In some compositions described herein, the composition possesses a degree of friability such that no more than, or exactly, 1% or 0.66% loss results after a friability test as per <USP 1216>. In some compositions described herein, the puck possesses a degree of friability that meets specifications provided by USP <1216>. In some compositions described herein, the compressed ground Cannabis material is milled or ground before being incorporated into the composition. In some compositions described herein, material is milled or ground and then passed through a mesh or a sieve before being incorporated into the composition. In some compositions described herein, the mesh or sieve has a mesh size of 30, 60, or 120. In some compositions described herein, the mesh or sieve has an average opening size of about 0.595 mm, about 0.250 mm, or about 0.125 mm. In some compositions described herein, the mesh or sieve has a mesh size of 60. In some compositions described herein, the mesh or sieve has an average opening size of about 0.250 mm. In some compositions described herein, the composition is formed by compressing its components into a predetermined shape. In some compositions described herein, the shape is a puck shape. In some compositions described herein, the shape is predetermined to be received by a Cannabis vaporizer.

Described herein are methods of preparing a defined dose Cannabis puck comprising: (a) preparing or obtaining a ground or milled Cannabis plant material preparation comprising a defined dose of one or more of one or more cannabinoids selected from the group consisting of: (i) 0.1-100 mg tetrahydrocannabinolic acid (THCA), (ii) 0.1-100 mg tetrahydrocannabidiol (THC), (iii) 0.1-100 mg cannabidiolic acid (CBDA), and/or (iv) 1-100 mg cannabidiol acid (CBD); (b) compressing the preparation of a) at 300-1500 PSI; and (c) recovering a defined dose Cannabis puck. In some methods described herein, step a) includes measuring and adjusting each cannabinoid to within +/− 5% of its defined amount. In some methods described herein, the maximum temperature of the preparation during compression is maintained below about 105° C. In some methods described herein, the maximum temperature of the preparation during compression is maintained below about 150° C. In some methods described herein, the compressing in step b) is carried out for a duration of from about 1 second to about 120 seconds. In some methods described herein, the puck comprises: THCA in an amount between 5-165 mg; THC in an amount less than 1.0 mg; and CBDA in an amount between 0.1-70 mg, and the puck has a total mass 100-500 mg. In some methods described herein, the puck comprises: THCA in an amount less than 5.0 mg; THC in an amount between 1-5 mg or between 5-135 mg; and CBD in an amount between 0.1-70 mg, and wherein the puck has a total mass 100-500 mg. In some methods described herein, the puck comprises: THCA in an amount less than 1.0 mg; THC in an amount less than 1.0 mg; and CBD in an amount between 5-90 mg, and wherein the puck has a total mass 100-500 mg. In some methods described herein, the Cannabis in the preparation of a) has not been previously exposed to accelerated dehydration at greater than about 100° C. In some methods described herein, following step b), the preparation is heated at a temperature above about 105° C. but below about 150° C. for a duration of about 5 minutes to about 30 minutes. In some methods described herein, prior to step a), the Cannabis preparation has been heated to a temperature above about 105° C. but below about 150° C. for a duration of about 5 minutes to about 30 minutes. In some methods described herein, immediately following step b), the preparation is cooled to a temperature below 10° C. Some methods described herein further comprise after step c), packaging the puck individually in a blister pack impermeable to gas exchange. Some methods described herein further comprise after step c) packaging the puck in a re-sealable multi-puck package impermeable to gas exchange. In some methods described herein, the compressing step is performed in a compression mold shaped to provide a signifier embossed on the puck which provides a visual indication of information on the defined dose of selected cannabinoids in the composition, and/or when the process comprises a further step of laser engraving a signifier on the puck which provides a visual indication of information on the defined dose of selected cannabinoids in the composition. In some methods described herein, the composition comprises packaging for containing the puck, the method further comprising a step of disposing the composition in packaging configured to associate the puck with a signifier which provides visual information on the defined dose of selected cannabinoids in the composition. In some methods described herein, the Cannabis plant material includes material derived from one or more members of a plant variety selected from the group consisting of Cannabis sativa, Cannabis indica, Cannabis ruderalis, and hybrids thereof. In some methods described herein, the Cannabis plant variety is Cannabis indica. In some methods described herein, the Cannabis plant material is prepared from Cannabis inflorescence. In some methods described herein, the preparation of step a) further comprises additives selected from among terpenes, terpenoids, puck stabilizers, humectants, vaporization aids, fillers and flavours. In some methods described herein, the Cannabis preparation in a) has been previously ground to sieve through a mesh of not larger than 1.5 mm in any surface dimension, and/or through a 30 mesh sieve, a 60 mesh sieve, or a 120 mesh sieve. Some methods described herein, further comprise (d) finishing the puck to provide a high gloss surface. In some methods described herein, the ground Cannabis preparation includes about 0.1 to about 1.0 grams total mass.

The pucks described herein can preferably be pressed into standardized amounts through compaction. These pucks provide a defined dose of selected cannabinoids and preferably are associated with a signifier which identifies such defined dose for the consumer. The pucks can be packaged individually in a blister pack or in a multi-puck pack. The pucks may identify a defined dose for a user or consumer by including a signifier that is laser burned onto the puck, for example setting out a defined dose of one or more active ingredients included in the puck.

### Incorporatioin by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flowchart depicting an exemplary method of forming a defined dose Cannabis puck.

FIG. 2 depicts an exemplary defined dose Cannabis puck in accordance with the current disclosure.

FIG. 3 depicts an exemplary defined dose Cannabis puck in accordance with the current disclosure.

FIG. 4A depicts an isometric view of an exemplary defined dose Cannabis puck in accordance with the current disclosure.

FIG. 4B depicts a side view of an exemplary defined dose Cannabis puck in accordance with the current disclosure.

FIG. 5A depicts a transparent view of an exemplary defined dose Cannabis puck in accordance with the current disclosure.

FIG. 5B depicts isometric view of an exemplary defined dose Cannabis puck in accordance with the current disclosure.

FIG. 6 depicts an exemplary defined dose Cannabis puck in accordance with the current disclosure.

FIG. 7 depicts an exemplary defined dose Cannabis puck in accordance with the current disclosure.

FIG. 8A and FIG. 8B depict an exemplary defined dose Cannabis puck in accordance with the current disclosure, fitted to custom fit into a cannabis vaporizer.

FIG. 9A and FIG. 9B depict a Cannabis vaporizer chamber and the defined dose Cannabis puck of FIG. 7 in cross-section.

### DETAILED DESCRIPTION OF THE INVENTION

Unpredictability is a hallmark of Cannabis. It starts with the species. The most common varieties, also referred to as chemical varieties or chemovars, worldwide, Cannabis sativa, Cannabis indica and Cannabis ruderalis, have distinct but overlapping ranges of cannabinoids. Over 100 cannabinoids may be found in these plants. Varieties and strains are continually crossed and or hybridized, generating different cannabinoid ratios. Further, the cannabinoid ratios within a single variety can be influenced by the conditions of cultivation, especially light cycle, temperature, soil condition, nutrient availability, and pathogen exposure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Harvesting and processing of Cannabis provides further room for variation. Every farmer knows the challenge of deciding when a crop is ready for harvest. Timing of harvest will influence cannabinoid amounts and ratios within a single variety. The degree of drying and/or curing the harvest will further influence the amount of cannabinoids by weight. Uncertainty also enters the process because the final product may be prepared exclusively from the inflorescence (also called flower or bud, being the plant part containing the highest cannabinoid concentrations), or alternatively some producers may feed other plant parts such as leaves and stem back into the final product as fillers.

The result is that a Cannabis product presented to a consumer can have, by weight, anywhere from 0% up to greater than 30% of selected cannabinoids, and the ratios between individual cannabinoids can be extraordinarily diverse.

Significantly, consumers cannot tell by visual inspection of a Cannabis preparation what amounts of the primary physiologically active cannabinoids tetrahydrocannabinol (THC), cannabidiol (CBD) and cannabigerol (CBG) are present. This uncertainty results from the fact that during the cultivation phase, Cannabis naturally synthesizes only the low potency precursors Tetrahydrocannabinolic acid (THCA), cannabidiolic acid CBDA and cannabigerolic acid (CBGA). These compounds convert respectively to THC (the primary psychoactive cannabinoid), and CBD and CBG (both significant non-psychoactive analgesic and anti-inflammatory cannabinoids) via decarboxylation. Decarboxylation may be induced by heating over 105° C. and/or by exposure to ultraviolet (UV) light. Whether the product has been so treated is not immediately apparent to a consumer. Among other things, this uncertainty creates a safety issue, because if the product is accidentally orally consumed by children or pets, there is no way of knowing if an alarming psychotropic event will result. (Gastric acids do not convert THCA to THC). See Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271.

Another area of relevant background pertains to two main methods of consuming Cannabis: smoking and vaping. Smoking is achieved with a wide variety of combustion devices, including cigarettes. The auto-ignition temperature of dried Cannabis is approximately 232° C. Ignition leads to much higher temperatures, all of which are sufficient to decarboxylate cannabinoids, which are then inhaled. “Vaping” is a method of vaporization whereby Cannabis is heated to a point below the auto-ignition point but above the THCA decarboxylation point (105° C.) and above the evaporation point of cannabinoids (e.g. approximately 157-226° C.). The user thereby inhales an evaporant that contains cannabinoids but does not contain combustion products.

Tools for making products of compressed Cannabis for consumption by smoking or vaping are provided by Storz & Bickel (Germany), which supplies tools for preparing by hand dosing capsules of compressed Cannabis suitable for vaping (SKU REF 09 45); and described by WO2016187696A1 in the name of Compressed Perforated Puck Technologies Inc. (Calgary, AB) which proposes a compressed vaporizer tablet and a method and instrument for making same.

Concerns about safety and unpredictability of Cannabis have historically been ignored by consumers. With increasing social and legal acceptance of Cannabis use, for example in North American jurisdictions including Canada and California, these concerns are likely to rapidly increase over time. This patent application pertains to Cannabis products with standardized, consistent amounts of cannabinoids so that users can find consistency and predictability which they have grown to expect in the field of alcoholic beverages.

The present invention relates to defined dose pucks of compressed Cannabis flower that are consumed by consumers particularly by direct vaporization, smoking, or integration into an edible matrix. The pucks comprise a defined dose of one or more cannabinoids and are preferably associated with a signifier of the defined dose.

This invention includes embodiments wherein the defined doses are distinct, and the uses are distinct. For example, embodiments include:
1) Low THC, high THCA Cannabis puck. This embodiment is a safety puck. It will have insignificant psychotropic activity on pets or children if accidentally orally ingested. If orally consumed it will be a non-psychotropic medicinal product. When vaped or smoked, the THCA converts to THC and delivers psychotropic effect.
2) Low THCA, high THC Cannabis puck. This puck requires a curing process (treatment at 105-150° C.) either of the initial Cannabis preparation, or of the final puck. It may optionally be used in orally consumed products to induce a psychotropic effect.
3) High CBD and negligible THCA or THC Cannabis puck. This puck is made from Cannabis varieties and cultivars which synthesize little or no THCA but abundant CBDA (which converts to CBD upon curing, vaping or combustion). Charlotte's Web is a suitable Cannabis cultivar to use in the preparation. This embodiment provides a non-psychotropic medicinal product when vaped or smoked.

The invention provides numerous improvements over the art, and may be associated with further alternative improvements which may be used in combination or alone to provide advantages for the puck, including but not limited to:
A step which includes measuring and adjusting each cannabinoid to within +/− 5% of its defined dose amount.
Association of the puck or its packaging with a signifier which provides an observer with information on the defined dose of selected cannabinoids in the composition.
Selection of the Cannabis from among Cannabis sativa, Cannabis indica, Cannabis ruderalis, and hybrids thereof.
Inclusion of additives selected from among puck stabilizers, humectants, vaporization aids, fillers and flavours. In some embodiments, the puck(s) include certain terpene or terpenoid compounds. For example, in some embodiments, pucks include added limonene, providing a lemon scent to the consumer. In other embodiments, pucks include added myrcene. Such pucks including added myrcene may be useful as sleep aids.
Adopting a puck physical form including a plurality of air channels of diameter not greater than 0.5mm disposed such that no portion of the puck is greater than about 0.5, 1, 1.5, 2, 2.5, or 3mm from an air surface.
Pucks having a high-gloss surface.
Pucks sealed individually in a blister pack impermeable to gas exchange, optionally in an inert gas environment.
A re-sealable multi-puck package impermeable to gas exchange.
Pucks having laser-engraved surfaces providing visual indicia to a consumer, for example indicating the amount of one or more psychoactive components included in the puck.

The methods of the invention improve upon the art by providing such steps as:
a precise and controlled method of grinding the Cannabis preparation to sieve through a mesh of not larger than 1.5 mm in any surface dimension; a method of compressing the preparation. Suitable compression pressures range from 10-2000 PSI;
temperature control of the compression step; and
a step of curing the product by temperature or UV treatment prior to compression or after.

Definitions

“Cannabis” as used herein includes all members of the Cannabis genus, including without limitation Cannabis sativa, Cannabis indica, Cannabis ruderalis, and hybrids thereof. “Cannabis” also includes Charlotte's Web and other high CBD, low THCA plant varieties.

“Cannabis inflorescence” means a cluster of flowers on a branch or a system of branches. An inflorescence is categorized on the basis of the arrangement of flowers on a main axis and by the timing of its flowering. Types of inflorescence may include solitary, spikes, racemes, and panicles. Cannabis is an example of a plant that forms racemes or “buds”. In a raceme, a flower develops at the upper angle (axil) between the stem and branch of each leaf along a long, unbranched axis.

“Cured” means harvested Cannabis which has been heated or cooked above 105° C. but below 115° C. for sufficient duration (30 minutes recommended) to convert essentially all THCA to THC by heat-induced decarboxylation.

“Uncured” means fresh harvest, unprocessed, or processed harvest which has not been exposed to temperature above 105° C. Product exposed to drying or accelerated dehydration which does not exceed 100° C. is considered uncured.

“Defined dose” means the dose of one or more active ingredients (typically cannabinoids) has been selected during the production process and is signified to a consumer by a signifier associated with the object.

“Friable” means a solid or semi-solid composition with enough structural integrity to maintain its shape and form under standard temperature, pressure and gravity conditions, but that may be crumbled or broken, partially or wholly, into fragments by forces or pressures not exceeding those of hand pressure or heat induced swelling.

“Gloss” is an optical property which indicates how well a surface reflects light in a specular (mirror-like) direction.

“High gloss surface” means, in the context of this invention, a surface treatment of a puck resulting in substantially higher gloss than that found with cured Cannabis inflorescence.

“Kief” refers to a composition predominantly containing isolated trichome nodules removed from the Cannabis inflorescence. Trichome nodules arise during the flowering stage on the outer surface of the inflorescence. They are enriched in cannabinoids. Kief may be removed after harvest by gently rubbing flowers (typically dried flowers) together, such as by hand or in a tumbling drum. Kief is captured on a 65-125 micron mesh. Immature trichomes will pass through such a mesh. Larger particles would be unwanted plant material. Grinding processes must consider whether the kief is to be separated or combined in the final grind.

“Psychotropic dose” means a dose of THC capable of affecting a user's mental state. Some people begin to notice an effect at doses greater than 1 mg THC.

“Less than a psychotropic dose” of THC means less than 1 mg THC.

“Puck” means a friable tablet having a relatively large surface to volume ratio.

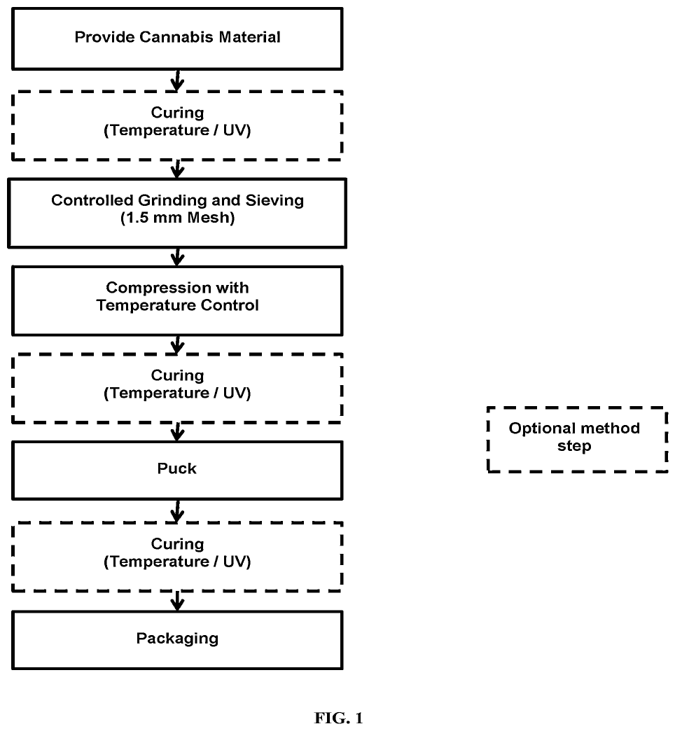

The methods of the invention which may be used to provide a composition of the invention will now be set out stepwise. The method steps and selected optional embodiments are generally set out in FIG. 1.

Grinding the Cannabis Preparation

The method of the invention requires a Cannabis preparation step wherein the Cannabis is ground into particles. The grinding step is a critical step that must be executed properly to achieve the preferred objects of the invention.

Grinding risks degradation of the product by generation of heat, by clumping of sticky materials, and by loss of material to the grinding instrument. All aspects must be carefully controlled to achieve superior results.

Preferably, Cannabis will be ground to sieve through a mesh of not larger than about 0.1 mm to about 3 mm, or any 0.1 mm increment therebetween, more preferably not larger than about 1.5 mm, in any surface dimension. In some embodiments, the sieve comprises 30, 60, or 120 mesh. In some embodiments, the sieve comprises an average opening size of 30, 60, or 120 mesh. In some embodiments, the sieve comprises an average opening size of about 0.595 mm, about 0.250 mm, or about 0.125 mm. In one embodiment, dried cannabis material is obtained for use in the grinding step. Cannabis material may include, without limitation, the leaves, inflorescences, flowers, or buds of one or more Cannabis plants. The grinding step may use any grinding method or methods, such as hand grinding, machine grinding, or use of a chipper or mulcher, provided that a consistent milled size product as homogenous as possible is generated without degradation. Degradation can occur through generation of heat during the grinding process and should be carefully controlled.

In another embodiment, the grinding step may grind the material to a particle size wherein 85-95% of the mass of particles have a maximum length less than about 0.1 mm to about 3 mm, or any 0.1 mm increment therebetween, preferably not larger than about 1.5 mm, and 5-15%, or any percentage increment therebetween, of the mass of the material are in particles have a length greater than about 0.1 mm to about 3 mm, or any 0.1 mm increment therebetween, preferably not larger than about 1.5 mm. Initial grinding may be followed by one or more filtering or sieving stages, for example to filter out stems or sticks. An illustrative mesh size for filtering or sieving the ground plant material may have an aperture size in the range of about 0.25 mm to 1.5 mm in its longest surface dimension. A coarse grinder having aperture larger than 1.5 mm is not preferred because it leads to air pocket trapping in the preparation which leads to undesirable uneven temperature during the compression step, and uneven temperature distribution during vaporization or combustion. A 60-mesh sieve is useful if a finer powder is desired to make the puck.

In some embodiments, the kief portion may be separated from the other plant material during the grinding step. In a preferred embodiment, the kief is not separated from the preparation used for compression, or if it has been separated, it is added back in. In general, the kief portion will be a large portion (by mass) of the smaller material, generally in the 65-125 micron range.

In some embodiments, additives may be added to the puck. In some embodiments, the puck(s) include certain terpene or terpenoid compounds. For example, in some embodiments, pucks include added limonene, providing a lemon scent to the consumer. In other embodiments, pucks include added myrcene. Such pucks including added myrcene may be useful as sleep aids.

It has been surprisingly observed that unground and unmilled Cannabis plant material is generally not suitable for preparation of friable pucks in accordance with the current invention. Regardless of compression pressure, pucks prepared from unmilled/unground material do not possess the desired characteristics such as friability. Additionally, milled or ground material demonstrates improved flowability. In general, the finer the grind, the better degree of flowability demonstrated by the material.

It has also been surprisingly observed that the content of ground or milled Cannabis preparations of Cannabis plant material contain lower proportions of cannabinoids than corresponding unground/unmilled plant material. It has also been surprisingly observed that the fraction of material that passes through a sieve, for example having a 30, 60, or 120 mesh, contains an elevated proportion of cannabinoids (e.g. THC) than does the input material. Hence, it has been surprisingly observed that sieving or meshing ground/milled Cannabis plant material in accordance with the present invention has the effect of cheaply and easily, without any chemical processing, increasing cannabinoid concentration in pure, additive free, Cannabis plant material (in the pass through fraction) and reducing concentration (in the retained fraction). It has also been surprisingly observed that by selecting an appropriate mesh size, one can selectively control the degree of concentration increase (in the pass through fraction) or decrease (in the retained fraction).

In a preferred embodiment, the Cannabis puck is prepared by obtaining or preparing a ground or milled Cannabis preparation. The milling may be performed by any suitable means, for example a household grinder or miller such as the Magic Bullet. The ground or milled preparation is then passed through a mesh or sieve. A fraction of material will then be retained on the sieve or mesh, while another fraction will pass through. The size of the particles which pass through will vary depending on the size of the openings in the mesh or sieve.

The concentration of one or more cannabinoids of interest (e.g. THC) is then measured in one or both of the pass-through and retained fractions. Based on the measured concentration, a target amount of retained fraction material, pass-through material, or a combination thereof, to be incorporated into the Cannabis puck is determined, such that the puck includes a desired defined dose of the one or more of one or more cannabinoids of interest. Finally, the desired amount of the material is formed into a puck by compression, for example at 0.2-5 atm or 300-1500 PSI.

### Dose Selection

The defined dose Cannabis pucks of the present invention have defined doses of one or more physiologically active compounds from Cannabis. In some embodiments, the pucks of the present invention have defined doses of one or more cannabinoids. Preferred defined dosages apply to cannabinoid compounds including, but not limited to: THC, THCA, CBD and CBDA. In some embodiments, the pucks may comprise a defined dose selected from the following ranges: about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THCA THCA, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the pucks comprise about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg of one of the foregoing compounds. In some embodiments, the pucks of the present invention have defined dosages for more than one of the foregoing compounds. For example, in some embodiments, the pucks comprise from about 0 mg-1 mg, or any 0.1 mg interval therebetween THC, about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the pucks comprise from about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the compositions are substantially free of THC-type cannabinoid compounds. For example, in some embodiments the pucks comprise from about 0 mg-1 mg, or any 0.1 mg interval therebetween THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD.

In some embodiments, the pucks described herein comprise an “effective” amount of one or more of the cannabinoid ingredients described herein. The term “effective amount” refers to an amount of the one or more cannabinoid ingredients sufficient to induce a change in an individual user. An effective amount also means an amount of the one or more cannabinoid ingredients that is needed to provide a desired level of cannabinoid(s) in the bloodstream of an individual user to provide an anticipated physiological response. An effective amount of a cannabinoid ingredient can be administered in one administration, or through multiple administrations of an amount that total an effective amount, preferably within a 24-hour period. It is understood that the effective amount can be the result of empirical and/or individualized (case-by-case) determination on the part of the individual user. For example, a therapeutically effective amount of said one or more cannabinoid ingredients may be in the range of about 1 mg to 2,000 mg, or any 1 mg or 10 mg interval therebetween total cannabinoids per day.

In some embodiments, an effective amount of said one or more cannabinoid ingredients may be in the range of about 1 mg-5 mg, or any 1 mg or 0.1 mg interval therebetween per day. For example, for an adult, about 1-2 mg, or 0.1 mg interval therebetween, a day total cannabinoids may provide a very low end dose below the psychoactive threshold.

In some embodiments, an effective amount of THC may be in the range of about 5 mg-90 mg, or any 1 mg interval therebetween. For example, most vapers inhale about 10 to 30 mg of THC to establish a mild, temporary, psychoactive effect.

In some embodiments, a composition of the present invention may comprise THCA in an amount between 5-165 mg,THC in an amount less than 1.0 mg, and CBDA in an amount between 0.1-70 mg, and have a total mass of 100-500 mg.

In some embodiments, a composition of the present invention may comprise THCA in an amount less than 5.0 mg, THC in an amount between 5-135 mg, and CBD in an amount between 0.1-70 mg, and have a total mass of 100-500 mg.

In some embodiments, a composition of the present invention may comprise THCA in an amount less than 1.0 mg, THC in an amount less than 1.0 mg, and CBD in an amount between 5-90 mg, and have a total mass of 100-500 mg.

In some embodiments, an effective amount of CBD for treating disorders such as pain, nausea, chronic pain conditions may be in the range of about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg per day. Preferably, the amount of CBD may be about 50 mg per day. For example, a recommended CBD serving standard may be about 25 mg of CBD taken twice a day.

In some embodiments, an effective amount of THCA may be in the range of about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg.

The therapeutic effects induced in an individual can be somewhat predictable but may vary from one individual to the next. The precise amount of cannabinoids required to induce an effect in an individual will depend upon numerous factors, e.g. type of cannabinoid(s), activity of a composition, intended use (e.g. number of doses per day), individual user considerations, methods of consumption, and others, which can readily be determined by one skilled in the art.

An achievement of the invention is that by using the composition of the invention, users and medical advisors for the first time have knowledge of the exact doses they are employing.

### Analytical Testing to Establish the Defined Dose

Two key analytical steps for the invention include:

1. Determining amounts of cannabinoids, especially THCA, THC, CBDA, and CBD of the Cannabis preparation prior to compression of an individual puck; and
2. Determining amounts of cannabinoids, especially THCA, THC, CBDA, and CBD after compression in the final Cannabis puck product.

For either step, any chemical analytical method may be employed to determine the amount of the cannabinoids. Many methods are available to those skilled in the art, such as those found in Thomas, B F and El Sohly, M 2015 “The Analytical Chemistry of Cannabis: Quality Assessment, Assurance, and Regulation of Medicinal Marijuana and Cannabinoid Preparations” (Elsevier). See also Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271; and Wang et al. (2017) Quantitative Determination of Cannabinoids in Cannabis and Cannabis Products Using Ultra-High-Performance Supercritical Fluid Chromatography and Diode Array/Mass Spectrometric Detection. J Forensic Sci.;62(3):602-611). A particularly recommended approach is found at Mudge et al. (2017) Anal Bioanal Chem (2017) 409:3153-3163 DOI 10.1007/s00216-017-0256-3.

The two key analytical steps are applied in a similar but distinct fashion in the process.

For example, prior to compression, the Cannabis preparation must contain the desired amounts of the selected cannabinoids to result in a puck having the desired defined dose. Due to the unpredictability of cannabinoids in the Cannabis plant that may result from strain variety, cultivation and harvesting conditions, the amount of cannabinoids per unit mass is never guaranteed. Nor are the relative ratios of cannabinoids. The first analytical step therefore will be to harvest and grind the “process batch” and to perform a batch assay to ascertain amounts of all relevant cannabinoids in a representative sample. The representative sample and measurement technique must be sufficient to represent all samples of the process batch within the degree of variability tolerated by the overall process, which as described below is +/− 5% of the defined dose of each cannabinoid.

Having identified the cannabinoid amounts of the process batch, it will need to be adjusted with filler to obtain the desired final amounts of cannabinoids for the preparation. In the method of the invention, the “filler” will be selected from among ground Cannabis preparations having known cannabinoid amounts which are different from the process batch. The operator will have available a series of “filler batches” with relatively higher or lower amounts and ratios of cannabinoids. The batch assay will allow the operator to identify by simple algorithm which amount of which filler batch(es) are required to obtain the final defined dose for the preparation before compression.

An alternative method to adjust the cannabinoid amounts of the process batch is to add cannabinoid concentrates (having predominantly aqueous carriers) and/or cannabinoid oils (predominantly lipid carriers). The operator will have available a variety of cannabinoid concentrates and/or oils of known cannabinoid concentrations (such as may be prepared by those skilled in the art). Again, by simple algorithm the operator can determine which amount of which concentrate or oil is required to bring the process batch into conformity with the desired dose of the final product. The concentrate or oil is contained in a liquid volume which, when mixed with the process batch, is fully absorbed by the dried Cannabis and does not cause clumping or sticking prior to compression. Such a composition may be considered an enriched Cannabis product.

After the filler batches are selected, they will be added, combined, and mixed thoroughly with the process batch. The final compression ready preparation may also be batch assayed to ensure the defined dose cannabinoids are present in the proper mass. Any final preparation which is not within tolerance levels is discarded or re-processed until desired cannabinoid levels are obtained. The final tolerance level is within 5%, preferably within 2% and most preferably within 1% of the desired in-going amount of each cannabinoid in the preparation.

The second analytical step is performed after the compression has been completed. It may be performed before or after the packaging and labelling process. In the second analytical step, a representative sample of defined dose pucks are analyzed to confirm the amount and ratios of cannabinoids present. This quality control step confirms whether the temperature and UV exposure conditions of the process have been properly observed. The chemical analytical tools employed may be the same or different from those employed in the first analytical step. A dissolution step will also be required to ensure the friable puck is suitably degraded so that it may be fully analyzed without loss to clumping or due to un-extractable particle sizes.

It is also noted that since moisture content is relevant to the flowability and stickiness of the ground Cannabis preparation prior to compression, the process operator will sometimes seek to determine and possibly adjust its moisture content. As the skilled person in the art will be aware, the moisture content of a Cannabis preparation may be obtained by Karl Fischer titration, following extraction of the vaporisable material in a suitable solvent such as methanol, or by heating the vaporisable material in a Stromboli sample oven. The Karl Fischer titration method is described in Fischer, K., Angew. Chem. (1935) 48 (26): 394-396.

### Compression Step

In one embodiment, the invention provides a method of producing a Cannabis puck by compressing loose plant source material into a compressible state. A general description of a Cannabis tablet press method is provided in patent application WO2016187696A1 and needs not be repeated here.

Herein we describe the further information required to describe and enable the claimed invention.

In some embodiments, a mechanical, pneumatic or hydraulic press may be used to provide sufficient compression force and desired ambient parameters to produce the Cannabis pucks described herein. Preferably, a hydraulic press will be used to produce compressed pucks which are suitable for use with a vaporization device. The compression force used will depend ultimately on the characteristics desired for the product. For example, the compression pressure may depend on the desired product characteristics for use in vaporization. In some embodiments, a compression pressure of about 0.2 atm to 5 atm may be used. In other embodiments, the compression step may be selected from 100 PSI to 4500 PSI. The preferred range is selected based on the compressibility of the cannabis plant matter, and the desired hardness of the resulting puck. In some embodiments compression pressures may be 300-1500 PSI, or for less compressible material, 1000 PSI may be preferred. In some embodiments, compression pressures may be 300-1500 PSI.

Ensuring that the proper amount of force is applied at the bottom of the stroke is crucial to avoid over exertion on the Cannabis preparation, and to avoid ripping and potential damage to the die and press. Those skilled in the art can select compression pressure and time optimized to generate a puck of desirable characteristics for use in vaping, or smoking, as desired by the consumer. In preferred embodiments, the pressure applied is maintained at an amount below that required to separate resin from plant material. Characteristics of the final friable puck are evenness of density and resistance to crumbling. The temperature resulting from compression and duration of the compression must be sufficient to soften lignins and heat oils such that upon cooling the puck retains the form of the die mold and is sufficiently durable for packaging and transporting to the consumer. Friability of resulting pucks may be assessed by means known in the art, and as described in the Examples further below. The inventors are aware that an aim of the invention is to generate pucks of acceptable hardness, including high hardness, but it should be understood that low hardness (high friability and instability of the puck structure during storage/transport) is a less desirable feature that should be avoided.

One important criterion regarding the hardness of the resulting puck is that for use in vaporization, the puck must rapidly heat throughout the entire core as heated air is drawn around the puck (and through the puck if it has channels). Compression must drive out air pockets and generate material to material contact such that entire puck is at a density such that when consumed it heats through in under 5 seconds, preferably under 3 seconds and most preferably under 2 seconds when exposed to air at a vape temperature of 226° C. Those skilled in the art can determine the compression temperature and time sufficient to achieve this level of successful heat transfer through the puck.

After compression, the puck is recovered from the die mold and allowed to cool to room temperature.

Non-limiting optional aspects of the method of the invention include:
Duration of compression: Compression will be exerted preferably from about 1 second to about 120 seconds.
The Cannabis preparation may be uncured, having not been previously exposed to accelerated dehydration at greater than about 100° C.
Where cured prior to compression, the Cannabis preparation may have been heated at above about 105° C. but below about 150° C. for a duration of about 5 minutes to about 30 minutes.
In a further alternative, curing takes place post-compression. In such an embodiment, after compression, the puck is heated at above about 105° C. but below about 150° C. for a duration of about 5 minutes to about 30 minutes.

Cooling steps may be employed throughout the process. In one embodiment, the die mold itself is cooled to avoid overheating the preparation during compression. In another embodiment, immediately following compression, the recovered puck is immediately cooled to below 10° C.

Critical temperatures for selecting process temperature control may be identified from the following list:

| Process | Temperature |
|---|---|
| Accelerated dehydration (drying) | Up to 95° C. |
| THCA decarboxylation to THC | 105° C. or higher |
| Curing of Cannabis flower (baking) | 105-113° C. for |
| | 30-45 minutes or longer |
| Evaporation of | 126° C. |
| Aromatic terpenoids | |
| THC | 157° C. |
| CBD | 160-180° C. |
| CBN | 185° C. |
| Efficiency of cannabinoid | |
| evaporation during vaporization | |
| “about half efficiency” | 150-180° C. |
| “highest efficiency” | 226° C. |
| Combustion/Auto-ignition | About 232° C. |
| temperature of dried Cannabis | and higher |
| Smoking of Cannabis | 232-1200° C. |

### Puck Mass and Shape, Air Holes and Edges

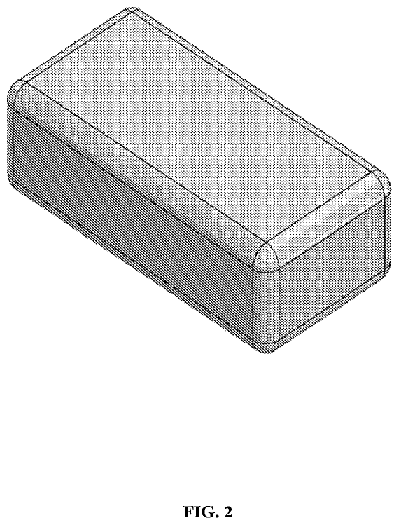

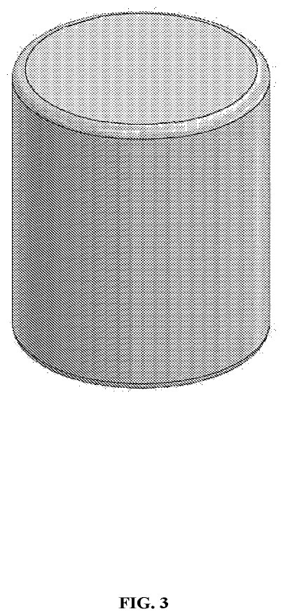

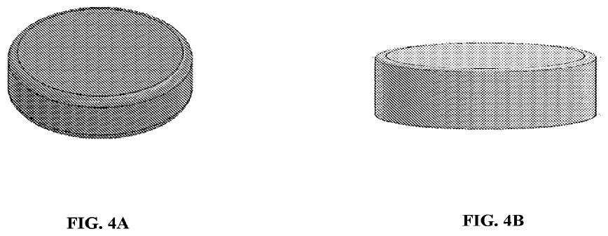

The compressed puck may be of any shape, such as oval, spherical, cylindrical, conical, cubic, rectangular, and the like. The shape of the compressed puck may be designed to accommodate a device, such as a vaporizer, a pipe, a bong, a “oneie,” and the like. Examples of a variety of shapes which are used to fit the cannabis bowls of such devices are provided in FIG. 2, FIG. 3, FIG. 4A and FIG. 4B. The finished total mass of the compressed Cannabis pucks of the invention ranges from about 0.05 g to about 1.0 g.

It will be appreciated that the amount of plant source material and the dimensions of the puck are not meant to be limiting. Puck sizes cover a range of options useful for a private single low dose user, to use of a single puck by a larger group of individuals and/or at higher doses. For example, the diameter of the puck may be about 5 mm to 20 mm and the thickness may be about 1 mm to 10 mm. The pod may be wider or narrower, or thinner or thicker as may be desired.

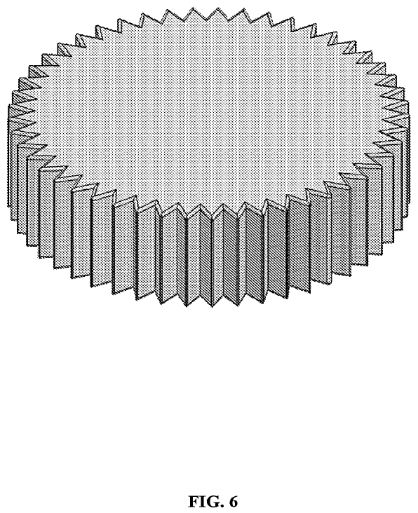

The edges of each puck can be shaped to provide greater surface area to enhance heat transfer. For example, pucks may have indentations or ridges along the outer side of each puck, so as to further increase the surface area of the puck. An example is shown in FIG. 6. A larger number of ridges can increase the rate of vaporization of the compressed plant source material. Generally the size and shape of the puck, and its edges, will be determined by the die mold selected for use during the compression step.

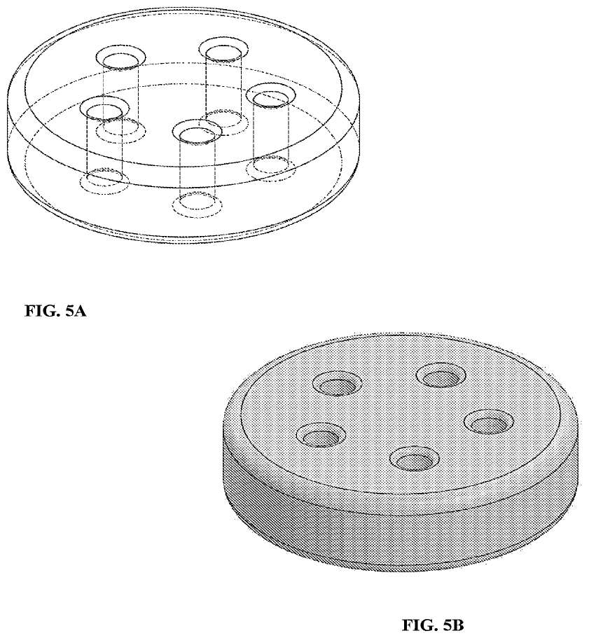

Optionally, a plurality of air channels of diameter not greater than 0.5mm are disposed such that no portion of the puck is greater than 2mm from an air surface. An example is shown in FIG. 5A and FIG. 5B. Patent application WO2016187696A1 provides a variety of methods of making air channels. Other methods are known to those in the art.

A variety of surface textures may be employed from rough to smooth, including high gloss coating discussed below.

Preferred shapes are those designed to fit in the bowls of leading vaporizer devices such as the Firefly 2 (thefirefly.com), Crafty (Storz & Bickel), Mighty (Storz & Bickel), or others including the DaVinci IQ, PAX 3, Arizer Solo 2 & Air 2, Hydrology9 by Cloudious9, Ghost MV1, Atmos VICOD 5G, and the Atmos Jump. Additional popular vaporizers for which pucks of the invention may be designed include the Airvape XS (Apollo), Arizer Air II, Arizer Extreme Q, Arizer Go ‘ArGo’ (all from Arizer), G-Pen Elite, G-Pen Pro and G-Pro (all from Grenco Science), the Boundless CF, CFV or CFX (Boundless), Ascent (DaVinci), Haze V3.0 (Haze Tech), Volcano Classic, Volcano Digital and the Volcano Plenty (Storz and Bickel).

Pucks may be embossed with a signifier such as a logo or design on at least one side of the puck. Signifiers include a barcode, or another type of machine readable code to identify the particular type of puck. Embossing of the puck with a signifier may take place contemporaneously with, or subsequent to, compression.

### Additives

In certain embodiments, the ground Cannabis preparation used in compression will contain non-Cannabis additives. A wide range of additives may be employed. Some additives may be useful as stabilizers or binders for the puck. Others may be humectants, vaporization aids, and diverse fillers known in the art. Excipients including but not limited to Ceolus KG and lactose monohydrate, when mixed with dried plant material, can assist with the compression step, and enhance desirable qualities of the resulting pucks.

A humectant is a hygroscopic substance that has an affinity to form hydrogen bonds with molecules of water and is used to produce a visible exhaled aerosol (i.e. vapour) when the product is in use. Suitable humectants for inclusion in a vaporisable material according to the present invention include propylene glycol, also known as 1,2-propanediol or propane-1,2-diol and having the formula C3H8O2 or HO—CH2—CHOH—CH3, and glycerol, also known as glycerine and having the formula C3H8O3. In a preferred embodiment, the humectant is propylene glycol. The present invention provides a Cannabis preparation having a moisture content of from about 3 to 5 wt %, and further comprising a humectant in an amount of at least 20 wt %.

A wide variety of flavouring agents, scents, perfumes and colouring agents, in addition to terpene additives such as limonene and myrcene, may also be employed in certain embodiments.

### Packaging Step

After the puck is recovered from compression, delivery to market is achieved by

a. packaging the puck individually in a blister pack impermeable to gas exchange; or

b. packaging the puck in a re-sealable multi-puck package impermeable to gas exchange.

The Cannabis pucks are preferably provided in a sealed package, which functions as an absolute barrier enabling the moisture content and flavour to be retained over time. The term “sealed package” refers to a gas-impermeable container having a hermetic closure which in the context of the present invention includes a blister pack. The Cannabis pucks may be individually sealed and packaged in blister packs. The blister packs may be designed to be child resistant and/or senior friendly in order to increase safety and convenience. While physically protecting the pucks, the blister pack controls humidity and is impermeable to gas exchange thereby maintaining freshness and enhancing the shelf life of the pucks.

Examples of the substantially gas exchange impermeable packaging include, but are not limited to, Al/A1 blister, and Al-polychloro-3-fluoroethylene homopolymer/PVC laminate blister. Alternatively, the sealed package may be a re-sealable multi-puck package impermeable to gas exchange. Such packages may be adapted from those known in the art by those skilled in the art to accommodate Cannabis pucks of the invention. Ideally, the recovered puck should be processed and packaged as quickly as possible to ensure that moisture is not lost from or absorbed into the composition.

Optionally, the packaging material is selected from materials which block some or all transmission of ultraviolet radiation. Use of such material will delay or prevent decarboxylation of cannabinoids such as THCA and CBDA, thereby preserving the defined dose characteristics of puck of the invention during storage and shelf exposure.

Pucks are expelled from production into the open blister cavities. Cavity depth and shape must be suitable for the puck. The open blister cavity is then sealed with a gas impermeable membrane to maintain quality of product and to reduce dehydration, rehydration or oxidation. To eliminate oxidation altogether, the packaging may be performed in an inert gas atmosphere. Optionally the blister is packed in an inert gas atmosphere such as nitrogen gas comprising little or no oxygen. To achieve this objective, the final sealing step of the packaging method may be operated in the inert gas atmosphere in a gas enclosure protected from ambient air.

### Association with Signifier

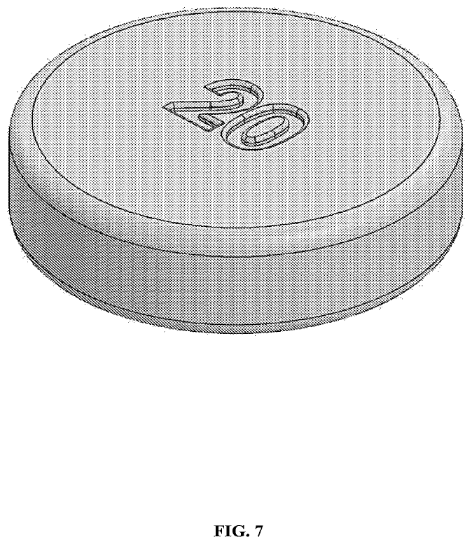

The method and composition of the invention associates the Cannabis puck with a signifier which allows the consumer to determine the defined dose of selected cannabinoids therein. A “signifier” means a visual mark or symbol that the consumer recognizes as referring to a specific defined dose. The signifier chosen may have elements of meaning, such as a number and unit, (e.g. “5 mg” or “10 mg” or simply “5” or “10”) or it may be an abstract signifier, where its meaning, in terms of defined dose, can be determined by reference to a standard. The meaning may be determined directly by the consumer or indirectly via a device. An example of a Cannabis puck with signifier “20” is shown in FIG. 7.

The signifier may be associated directly with the Cannabis puck during or after compression by such means as embossing, or by colour, pattern or shape feature. Alternatively the signifier may be associated with the packaging. The packaging may include signifiers directly interpreted by consumers or signifiers which are machine readable codes. In all embodiments, the signifier allows the consumer to determine the defined dose of selected cannabinoids therein.

### Optional Finishing Step Prior to Packaging

Gloss is an optical property which indicates how well a surface reflects light in a specular (mirror-like) direction. It is one of important parameters that are used to describe the visual appearance of an object. The factors that affect gloss are the refractive index of the material, the angle of incident light and the surface topography. Gloss provides an attractive commercial feature for consumers.

The invention includes a high gloss Cannabis puck, and methods of making it. Several general methods to achieve a glossy finish are known in the art which may be applied to the invention herein. A flash gloss process may be used. This process requires heating the exterior surface to softening point of cellular lignins at a maximum of 80-140° C. for 5 seconds or less, followed by slow cooling (10° C. per minute) for 5-10 minutes. The cooling lignins will crystallize on the outer surface of the puck to provide a glossy finish.

Alternatively, a glossy coating may be applied to the outer surface. It may be applied by a painting application. Or it may be provided by applying a fine layer (0.01 to 0.005 mm) of a crystallizable material to an exterior surface of the puck followed by a flash gloss process. The coating applied in such cases must be suitable for use with vaping, or smoking, as the case may be. It may optionally comprise kief or other cannabinoid extracts such as shatter, oils or waxes.

### EXAMPLES

Defined dose cannabis pucks with standardized, consistent amounts of cannabinoids were manufactured. Examples of pucks containing either THCA or CBDA are described below.

Equipment

Milling Equipment-Magic Bullet

R&D Single Punch Puck Press

Weight: 751bs.

Pressure Gauge: Can go up to pressure of 5000 PSI

Tooling: ⅜″ round concave tooling, including 1 316S steel upper punch. 1 316S steel lower punch and 1 3165 steel die tooling

Vernier Calipers

Cannabis Preparation: Milling and Sieving

Cannabis is prepared according to the standard methods disclosed in the specification above. Milling and sieving may be employed to develop a finer grain of product for puck manufacturing. Surprisingly, as is shown below, milling and sieving may lead to increased potency of cannabis in the finer portions of the ground product.

Mesh sizes referred to herein are standard US mesh size designations. The mesh number corresponds to number of openings in one square inch of screen, e.g. a 100-mesh screen has 100 openings. As the mesh size increases, the size of the particles decreases. Higher numbers equal finer material. A 60 mesh sieve has a mesh opening of 250 μm. With a 60 mesh sieve, milled as indicated, 66.5% by mass is retained on the sieve and 33.5% by mass passes through onto the sieve pan.

Also, for general reference in the examples below, a cannabis puck of the invention may be described as containing e.g. 56 mg THCA (50 mg THC). This convention is based on the fact that THCA has a molecular weight of 357.47 g/mol. When decarboxylated (by vaping or smoking) the THCA converts to THC (molecular weight 314.45 g/mol). As such, a puck of the invention with 56 mg THCA may also be described as providing 50 mg THC. This convention is applied in Examples 1 and 2 below.

In the following examples, where total THC is marked as “total %THC*” or “mg per puck THC*” it has been calculated by converting THCA (in source material) to the corresponding amount of THC that would result upon complete decarboxylation, then adding the amount of THC (already decarboxylated) found in the source material.

Example 1

### Prototype of Pink Kush Flower Puck Formulation Containing THCA at Theoretical 56 mg Dose

A certificate of analysis determined the THCA potency of the original Pink Kush dried flower sample to be 23.34%. This material was used to prepare pucks having a theoretical dose of 56 mg THCA. Prior to the manufacturing of puck the flower was dried and then milled using milling equipment which is generally used for milling plant materials. The flower was milled for approximately 1-3 minutes. This was followed by sieving the material through a) 30 mesh sieve (595 μm opening) b) 60 mesh sieve (250 μm opening) c) 120 mesh sieve (125 μm opening). During the sieving process, milled material was pushed through 30 mesh, approximately 19% retained on 30 mesh sieve, 81% passed through the 30 mesh sieve. On the 60 mesh sieve 59% of the flower material was retained and 41% passed through the 60 mesh sieve. With the 120 mesh sieve approximately 67% was retained on the 120 mesh sieve and 33% passed through the 120 mesh sieve. The material retained on each mesh and material which passed through the 30, 60, and 120 mesh was compressed into pucks.

The pucks were evaluated for potency. With an expected potency of 23.34%, pucks manufactured at a total mass of 239 mg were expected to contain theoretical dose of 56 mg THCA (50 mg THC).

Milled cannabis retained on mesh demonstrates reduced potency: The milled material retained on the 30 mesh and 60 mesh was compressed and provided a potency of 20-24 mg/puck of THCA. This is substantially lower than theoretical dose of 56 mg THCA (50 mg THC). Pucks manufactured with milled material retained on the 120 mesh had potency of 39 mg THCA and 35 mg THCA instead of 56 mg THCA. This could be the result of the milling process breaking up the flower material which led to a lower potency.

Cannabis pass-through material demonstrates normal or enhanced potency: Pucks manufactured with milled material and sieved through the 30 mesh had potency of 58 mg THCA and 52 mg THC which is within specifications. The pucks manufactured with material that passed through the 60 mesh had higher potency of 73 mg THCA and 65 mg THC which is approximately 30% increase in potency over the theoretical 56/50 mg doses. The milled material which was sieved through the 120 mesh had higher potency of 74 mg THCA and 67 mg THC which is approximately 30% increase in potency over the theoretical 56/50 mg doses.

A detailed description of the puck manufacturing and testing procedure is provided below.

### Sieving Example/Impact on Potency

Results-56 mg THCA Puck

| | Expected | Theoretical Fill | |
|---|---|---|---|
| Formulation | Potency (%) | Weight (mg) | Dose |
| Pink Kush | 23.34% | 239 | 56 mg THCA |
| (THCA) | | | |

Flower Milling

Approximately 10 g of dried flower was placed into a mill and milled for 1-3 minutes to finer powder. The milled flower was then pushed through a) 30 mesh sieve with a 595 μm opening b) 60 mesh sieve with a 250 μm opening and c) 120 mesh sieve 125 μm opening. The powder from the Pink Kush has a light green colour to it. The sieved material collected on the mesh and material collected in the sieve was used for the manufacturing of pucks.

| | Total | | Amount | % |
|---|---|---|---|---|
| Formulation | (g) | Sieve No. | Retain (g) | Retain |
| (THCA) | 9.69 | 30 | 1.81 | 18.7 |
| | | 30-Sieve | 7.88 | 80.5 |
| | | Pan | | |
| | 11.98 | 60 | 7.1 | 59.2 |
| | | 60-Sieve | 4.88 | 40.7 |
| | | Pan | | |
| | 10.55 | 120 | 7.03 | 66.6 |
| | | 120-Sieve | 3.52 | 33.3 |
| | | Pan | | |

S = Sieved material collected on pan
US = Unsieved material retained on mesh

After milling and putting the milled material through 30 mesh, approximately 19% of the flower material was retained on 30 mesh sieve, 81% passed through the 30 mesh sieve. On the 60 mesh sieve 59% of the flower material was retained and 41% passed through the 60 mesh sieve. With the 120 mesh sieve approximately 67% of the flower material was retained on the 120 mesh sieve and 33.3% passed through the 120 mesh sieve.

Potency Calculation for 56 mg THCA Puck

To manufacture 56 mg THCA flower using Pink Kush the calculation is as follows:

$$Potency of THCA = 23.34 \%$$

$$\begin{matrix}Puck Weight for 56 mg THCA = Defined Dose ( mg ) / Potency of THCA \\ = 56 mg / 0.2334 \\ = 239 mg puck weight\end{matrix}$$

Compression

The puck weight was chosen based on each puck being able to deliver 56 mg THCA, based on the potency calculation the pucks were compressed on the tablet press at a puck weight of 239 mg ±5% to deliver. The compression force used was 1000 PSI which gave a more robust puck with the required hardness and puck integrity. Three pucks were manufactured for material that passed through the 30, 60, and 120 mesh sieve; the material that was retained on the 30, 60 and 120 mesh sieve was also manufactured to compare/evaluate if there is any difference in potency.

56 mg THCA Pucks (Milled Flower-Retained on 30 Mesh Sieve)

| | Puck Weight | | | | |
|---|---|---|---|---|---|
| | (mg) | | | | |
| | Target 239 | | Dwell | Puck | Puck |
| | mg ± 5% | Compression | Time | Diameter | Thickness |
| Description | (228-251 mg) | Force | (sec) | (mm) | (mm) |
| Pink Kush | 240 | 1000 | Less | 9.76 | 3.40 |
| (THCA) | 240 | | than 3 | 9.65 | 3.58 |
| | 239 | | | 9.65 | 3.47 |
| Average | 239 | | | 9.69 | 3.48 |

56 mg THCA Pucks (Milled Flower-Retained on Sieve Pan after Pass through 30 Mesh)

| | Puck Weight | | | | |
|---|---|---|---|---|---|
| | (mg) | | | | |
| | Target 239 | | Dwell | Puck | Puck |
| | mg ± 5% | Compression | Time | Diameter | Thickness |
| Description | (228-251 mg) | Force | (sec) | (mm) | (mm) |
| Pink Kush | 239 | 1000 | Less | 9.68 | 3.40 |
| (THCA) | 246 | | than 3 | 9.71 | 3.58 |
| | 240 | | | 9.69 | 3.47 |
| Average | 242 | | | 9.69 | 3.48 |

56 mg THCA Pucks (Milled Flower-Retained on 60 Mesh Sieve)

| | Puck Weight | | | | |
|---|---|---|---|---|---|
| | (mg) | | | | |
| | Target 239 | | Dwell | Puck | Puck |
| | mg ± 5% | Compression | Time | Diameter | Thickness |
| Description | (228-251 mg) | Force | (sec) | (mm) | (mm) |
| Pink Kush | 231 | 1000 | Less | 9.69 | 3.15 |
| (THCA) | 240 | | than 3 | 9.68 | 3.31 |
| | 234 | | | 9.69 | 3.36 |
| Average | 235 | | | 9.69 | 3.27 |

56 mg THCA Pucks (Milled Flower-Retained on Sieve Pan after Pass 60 Mesh)

| | Puck Weight | | | | |
|---|---|---|---|---|---|
| | (mg) | | | | |
| | Target | | Dwell | Puck | Puck |
| | 239 mg ± 5% | Compression | Time | Diameter | Thickness |
| Description | (228-251 mg) | Force | (sec) | (mm) | (mm) |
| Pink Kush | 242 | 1000 | Less | 9.72 | 3.62 |
| (THCA) | 235 | | than 3 | 9.75 | 3.47 |
| | 228 | | | 9.57 | 3.31 |
| Average | 235 | | | 9.69 | 3.47 |

56 mg THCA Pucks (Milled Flower-Retained on 120 Mesh Sieve)

| | Puck Weight | | | | |
|---|---|---|---|---|---|
| | (mg) | | | | |
| | Target | | Dwell | Puck | Puck |
| | 239 mg ± 5% | Compression | Time | Diameter | Thickness |
| Description | (228-251 mg) | Force | (sec) | (mm) | (mm) |
| Pink Kush | 237 | 1000 | Less | 9.68 | 3.36 |
| (THCA) | 226 | | than 3 | 9.67 | 3.25 |
| | 238 | | | 9.68 | 3.47 |
| Average | 234 | | | 9.68 | 3.36 |

56 mg THCA Pucks (Milled Flower-Retained on Sieve Pan after Pass 120 Mesh)

| | Puck Weight | | | | |
|---|---|---|---|---|---|
| | (mg) | | | | |
| | Target | | Dwell | Puck | Puck |
| | 239 mg ± 5% | Compression | Time | Diameter | Thickness |
| Description | (228-251 mg) | Force | (sec) | (mm) | (mm) |
| Pink Kush | 228 | 1000 | Less | 9.70 | 3.22 |
| (THCA) | 234 | | than 3 | 9.73 | 3.26 |
| | 231 | | | 9.67 | 3.10 |
| Average | 231 | | | 9.80 | 3.28 |

Vernier calipers were used to measure the puck diameter and thickness.

### Packaging

The pucks were packaged into amber glass bottles and stored at 4° C.

### Potency Data

Potency of Flower Material (Unmilled and Unsieved)

| % THCA | % THC | Total % THC* |
|---|---|---|
| 23.8 | 0.54 | 21.4126 |

THCA and THC Data for 239 mg Puck (Retained on 30 Mesh)

| | | mg per puck | | mg per puck |
|---|---|---|---|---|
| | % THCA | THCA | % THC | THC* |
| | 10.2 | 24.4 | 0.4 | 22.3 |
| | 10.0 | 23.8 | 0.4 | 21.8 |
| | 8.6 | 20.6 | 0.3 | 18.9 |
| Average | 9.6 | 23.0 | 0.4 | 21.0 |
| SD | | 2.0 | | 1.8 |
| RSD | | 8.8 | | 8.8 |

THCA and THC Data for Milled Flower (Retained on 30 Mesh)

| % THCA | % THC | Total % THC* |
|---|---|---|
| 9.08 | 0.74 | 8.70 |

THCA and THC Data for 239 mg Puck (Retained on Sieve Pan after Pass 30 Mesh)

| | | mg per puck | | mg per puck |
|---|---|---|---|---|
| | % THCA | THCA | % THC | THC* |
| | 24.5 | 58.6 | 0.5 | 52.7 |
| | 23.9 | 57.1 | 0.5 | 51.4 |
| | 24.2 | 57.8 | 0.5 | 52.0 |
| Average | 24.2 | 57.8 | 0.5 | 52.0 |
| SD | | 0.7 | | 0.6 |
| RSD | | 1.2 | | 1.2 |

THCA and THC Data for Milled Flower (Retained on Sieve Pan after Pass 30 Mesh)

| % THCA | % THC | Total % THC* |
|---|---|---|
| 23.5 | 0.55 | 21.2 |

THCA and THC data for 239 mg Puck (Retained on 60 Mesh)

| | | mg per puck | | mg per puck |
|---|---|---|---|---|
| | % THCA | THCA | % THC | THC* |
| | 11.3 | 23.7 | 0.3 | 24.4 |
| | 11.4 | 23.9 | 0.3 | 24.6 |
| | 11.2 | 23.5 | 0.3 | 24.2 |
| Average | 11.3 | 23.7 | 0.3 | 24.4 |
| SD | | 0.2 | | 0.2 |
| RSD | | 0.9 | | 0.9 |

THCA and THC Data for 239 mg Milled Flower (Retained on 60 Mesh)

| % THCA | % THC | Total % THC* |
|---|---|---|
| 11.8 | 0.33 | 10.7 |

THCA and THC Data for 239 mg Puck (Retained on Sieve Pan after Pass 60 Mesh)

| | | mg per puck | | mg per puck |
|---|---|---|---|---|
| | % THCA | THCA | % THC | THC* |
| | 27.9 | 66.7 | 0.6 | 59.8 |
| | 30.5 | 72.9 | 0.6 | 65.4 |
| | 32.7 | 78.2 | 0.7 | 70.1 |
| Average | 30.4 | 72.6 | 0.6 | 65.1 |
| SD | | 4.7 | | 4.2 |
| RSD | | 6.5 | | 6.5 |

THCA and THC Data for Milled Flower

(Retained on Sieve Pan after Pass 60 Mesh)

| % THCA | % THC | Total % THC* |
|---|---|---|
| 29.9 | 0.64 | 26.9 |

THCA and THC Data for Milled Flower (Retained on 120 Mesh)

| % THCA | % THC | Total % THC* |
|---|---|---|
| 17.7 | 0.51 | 16.0 |

THCA and THC Data for 239 mg Puck (Retained on Sieve Pan after Pass 120 Mesh)

| | | mg per puck | | mg per puck |
|---|---|---|---|---|
| | % THCA | THCA | % THC | THC* |
| | 31.4 | 75.0 | 0.6 | 67.3 |
| | 30.8 | 73.6 | 0.6 | 66.0 |
| | 30.5 | 72.9 | 1.3 | 67.0 |
| Average | 30.9 | 73.9 | 0.8 | 66.8 |
| SD | | 0.9 | | 0.5 |
| RSD | | 1.2 | | 0.8 |

THCA and THC Data for Milled Flower (Retained on Sieve Pan after Pass 120 Mesh)

| % THCA | % THC | Total % THC* |
|---|---|---|
| 30.3 | 0.634 | 27.2 |

### Flower and Milled Flower Potency

The potency of the initial dried flower plant for Pink Kush had potency of 23.8% THCA (21.4% for THC), which is within specs based on dried flower certificate of analysis.

Milled material retained on the 30 mesh sieve which was not compressed into pucks gave THCA and THC potency of 9.1% and 8.7% respectively. The milled material which passed through the 30 mesh sieve gave potency of 23.5% THCA and 21.2% THC.

Milled material retained on the 60 mesh sieve gave THCA and THC potency of 11.8% and 10.7% respectively. The milled material which passed through the 60 mesh sieve gave potency of 29.9% THCA (26.9% THC).

Milled material retained on the 120 mesh sieve gave THCA and THC potency of 17.7% and 16.0% respectively. The milled material which passed through the 60 mesh sieve gave potency of 30.3% THCA (27.2% THC).

### Puck Potency

Pucks manufactured at a weight of 239 mg were expected to contain a theoretical dose of 56 mg THCA (50 mg THC). The milled material retained on the 30 mesh and 60 mesh was compressed and gave a potency of 20-24 mg/puck of THCA. This is significantly lower than the theoretical dose of 56 mg THCA. Pucks manufactured with milled material retained on the 120 mesh had potency of 39 mg THCA and 35 mg THCA.

Pucks manufactured with milled material and sieved through the 30 mesh had potency of 58 mg THCA (52 mg THC) which is within specifications. The pucks manufactured with material that passed through the 60 mesh had higher potency of 73 mg THCA (65 mg THC), an approximate 30% increase in potency. The milled material which was sieved through the 120 mesh had a potency of 74 mg THCA (67 mg THC), an approximate 30% increase in potency

56 mg THCA pucks were manufactured with the flower material. The flower was milled and then put through 30, 60, and 120 mesh sieves. The material retained on the mesh and the material retained on the sieves was compressed to evaluate if there was a difference in potency.

After milling and putting the milled material through 30 mesh, approximately 19% retained on 30 mesh sieve, 81% passed through the 30 mesh sieve. On the 60 mesh sieve 59% of the flower material was retained and 41% passed through the 60 mesh sieve. With the 120 mesh sieve, approximately 67% was retained on the 120 mesh sieve and 33.3% passed through the 120 mesh sieve.

The pucks were compressed at 1000 PSI, which gave acceptable hardness and integrity.

The milled material used for manufacturing the pucks retained on the 30 and 60 mesh sieves gave lower potency in comparison to the theoretical dose of 56 mg THCA (50 mg THC) by approximately 38%. However, the milled material retained on the 120 mesh sieve had slightly higher potency of approximately 39 and 35 mg, which was less than theoretical dose of 56 mg THCA (50 mg THC). It is important to note that milling the material did reduce the overall potency of the flower.

The milled material used for manufacturing of pucks which passed through the 30 mesh sieve had potency which was within specifications for theoretical dose of 56 mg THCA (50 mg THC).

The milled material used for manufacturing of pucks which passed through the 60 mesh sieve and 120 mesh sieve had potency which had higher potency by approximately 30% in comparison to theoretical dose of 56 mg THCA (50 mg THC).

Milling the flower material reduces the potency of the flower. However, milling and sieving the flower through the 60 mesh and 120 mesh sieve does increase the potency. Alternatively, the milled material can also be used for manufacturing pucks.

### Example 2

### A Prototype of a B27 Flower Puck Formulation Containing CBDA Amounts at Either Theoretical 20 mg or 45 mg Doses

B27 flower was first determined by analysis to comprise 9.53% (w/w) of CBDA. Prior to the manufacturing of puck(s) the flower was dried and then milled using milling equipment which is generally used for milling plant materials. The flower was milled for approximately 1-3 minutes. This was followed by sieving the material through a 60 mesh sieve which has a mesh opening of 250 μm. The resultant pass-through material obtained was very fine like powder material. The pucks were then compressed at theoretical doses of 20 mg and 45 mg CBDA.

The actual potency data showed higher potency than theoretical potency. During sieving, the plant material is removed from the flower and the trichome heads are retained which contain high concentration of CBDA/CBD. Hence, the overall potency of CBDA and CBD increased by approximately 10%, giving actual potency of 24 mg CBDA (22 mg CBD), 52 mg CBDA (49 mg CBD) in comparison to theoretical potency of 20 mg and 45 mg CBDA respectively

A prototype of a B27 flower puck formulation containing CBDA amounts at the theoretical 20 mg dose was manufactured. Prior to the manufacturing of puck(s) an additional step was performed. The flower was dried and then milled using milling equipment which is generally used for milling plant materials. Approximately 10 g of flower material was milled for approximately 1-3 minutes. This was followed by sieving the material through a 60 mesh sieve which has a mesh opening of 250 μm. Approximately 66.5% was retained on the 60 mesh sieve and 33.5% was pass-through material retained on the sieve pan. The resultant material retained on the 60 mesh sieve and the material which passed through the 60 mesh sieve was compressed at 1000 PSI at theoretical dose of 20 mg CBDA.

Cannabis retained on sieve demonstrates reduced potency: Pucks manufactured with material retained on 60 mesh gave average potency 8.0 mg CBDA (7.5 mg CBD) which is below theoretical dose of 20 mg CBDA. Based on the CofA (certificate of analysis), this is a decrease in potency by 60%. Pucks manufactured with material passing through 60 mesh gave average potency of 23.6 mg CBDA (22.2 mg CBD) which is an increase in potency by 15% for both CBDA and CBD.

The potency decreased further by milling the flower material and passing through a 60 mesh sieve; the fraction of material retained in the 60 mesh sieve demonstrated a 60% decrease in potency. However, the flower material that was milled, passed through the 60 mesh sieve, and retained on the sieve pan showed increase in potency by 15%.

Additional information for potency of the puck as result of the milling/sieving process at two dosage levels are described in detail below under the headings Study#1 20 mg CBDA Puck and Study#2 45 mg CBDA Puck.

### Results

### Study #1-20 mg CBDA Puck

| | Theoretical | | Theoretical | |
|---|---|---|---|---|
| | Potency (%) | | Fill Weight | |
| Formulation | (per CoA) | Lot No. | (mg) | Dose |
| B27 (CBDA) | 9.53% | 0143 | 210 | 20 mg CBDA |

### Milling of Flower

Approximately 10 g of dried flower was placed into a mill and milled for 1-3 minutes to finer powder. The milled flower was then pushed through a 60 mesh sieve with a 250 μm opening to a more fine powder. The powder for the B27 CBDA dried flower had a darker green colour to it. The sieved and milled material was used for the manufacturing of pucks. 66.5% was retained on 60 mesh sieve and 33.5% was sieved and retained on sieve pan.

| | Theoretical | | | Amount | |
|---|---|---|---|---|---|
| Formulation | Potency (%) | Lot No. | Sieve No. | Retain (g) | % Retain |
| B27 (CBDA) | 9.53% | 0143 | 60 | 6.31 | 66.5 |
| B27 (CBDA) | 9.53% | 0143 | Sieve Pan | 3.18 | 33.5 |

### Density of Flower Material

The density of flower material was measured by filling up a 10mL graduated cylinder and weight was recorded and the mL occupied by the flower was also recorded. Afterwards, tapped density was recorded by tapping the flower in the graduated cylinder 100 times and the change in volume was recorded. The tapped density demonstrates compressibility of the flower. A higher tapped density entails good compressibility whereas lower density indicates poor compressibility.

Density of Flower=1.48/8.5 mL=0.174 g/mL

Tapped Density of Flower=1.48 g/5.75 mL=0.257 g/mL

### Potency Calculation for 20 mg CBDA Puck

To manufacture 20 mg CBDA flower using B27 flower the calculation is as follows:

$$Potency of CBDA = 9.53 \%$$

$$\begin{matrix}Puck Weight for 20 mg CBDA = Defined Dose ( mg ) / Potency of CBDA \\ = 20 mg / 0.0953 \\ = 210 mg puck weight\end{matrix}$$

### Compression

The initial puck manufactured on the tablet press was at weight of 380 mg at compression force of 2000 PSI. The puck produced at this force was hard to break and therefore the pressure had to be reduced. The next puck was made at weight of 458 mg at force of 500 PSI and the puck was still too hard and not ideal for vaporizer since the assumption is that the if the puck is too hard it will not vaporize within the chamber. Puck was manufactured on tablet press at weight of approximately 500 mg with 300-1500 PSI compression force and this puck demonstrated acceptable hardness.

The desired weight of the puck was determined based on delivering 20 mg CBDA which gave a puck weight of 210 mg. The pucks were compressed at a puck weight of 210 mg ±5% to deliver 20 mg CBDA. The weight of each puck manufactured is provided in the table below.

### 20 mg CBDA (Milled B27 Flower 60 Mesh Sieve)

| | Puck Weight | | | | |
|---|---|---|---|---|---|
| | (mg) | | | | |
| | Target | | Dwell | Puck | Puck |
| | 210 mg ± 5% | Compression | Time | Diameter | Thickness |
| Description | (200-226 mg) | Force | (sec) | (mm) | (mm) |
| B27 | 210 | 300 | Less | 9.71 | 4.58 |
| (CBDA) | 203 | | than | 9.90 | 4.62 |
| | 207 | | 3 | 9.90 | 4.41 |
| | 204 | | | 9.87 | 5.09 |
| | 207 | | | 9.90 | 4.86 |
| | 202 | | | 9.95 | 4.73 |
| | 207 | | | 9.81 | 4.17 |
| | 210 | | | 9.89 | 4.57 |
| | 202 | | | 9.88 | 4.60 |
| | 206 | | | 9.58 | 4.41 |
| | 211 | | | 9.82 | 4.62 |
| | 210 | | | 9.77 | 4.26 |
| | 202 | | | 9.73 | 3.84 |
| | 212 | | | 9.87 | 4.44 |
| | 205 | | | 9.85 | 4.76 |
| Average | 207 | | | 9.83 | 4.53 |

No. of pucks manufactured 15

### Friability

Friability testing was performed on the 20 mg pucks and loss of 0.9% was obtained which is less than 1% and meets specifications provided by the guidelines of USP Chapter <1216>Tablet Friability (herein “USP <1216>”).

| | Weight | Weight | Friability |
|---|---|---|---|
| Description | Before (g) | After (g) | (%) |
| B27 | 1.010 | 1.001 | 0.9% |
| (CBDA) | | | |

### Packaging

The pucks were packaged into white opaque bottles with child resistant caps and stored at 4° C.

| | | Tare Weight | Net Weight |
|---|---|---|---|
| Description | Bottle # | (g) | (g) |
| B27- | 1 | 40.18 | 3.07 |
| 20 mg CBDA | | | |
| per puck | | | |

Potency Data

B27 210 mg Puck Data

| | | mg per puck | | mg per puck |
|---|---|---|---|---|
| | % CBDA | CBDA | % CBD | CBD* |
| | 11.9 | 24.633 | 0.74 | 23.134941 |
| | 11.2 | 23.184 | 0.7 | 21.781368 |
| | 11 | 22.77 | 0.695 | 21.40794 |
| Average | 11.3666667 | 23.529 | 0.711666667 | 22.108083 |
| SD | | 0.978243835 | | 0.908674932 |
| RSD | | 4.157609055 | | 4.110148003 |

The potency data for the theoretical B27 20 mg CBDA puck data gave an interesting result. The average potency of the B27 theoretical 20 mg CBDA puck tested to be 24 mg CBDA. The potency of CBD was calculated to be 22 mg CBD. The potency increased by approximately 10%.

Study #2-45 mg CBDA Puck

To evaluate compression of puck prototypes at puck weight of 472 mg and determine optimal compression force required to manufacture pucks with acceptable integrity.

| | | | Theoretical | |
|---|---|---|---|---|
| | Theoretical | | Fill Weight | |
| Formulation | Potency (%) | Lot No. | (mg) | Dose |
| B27 (CBDA) | 9.53 | 0143 | 472 | 45 mg CBDA |

### Milling of Flower

Approximately 10 g of dried flower was placed into a mill and milled for 1-3 minutes to finer powder. The milled flower was then pushed through a 60 mesh sieve to a more fine powder. The powder for the B27 flower has a darker green colour to it. The sieved and milled material was used for the manufacturing of pucks.

### Potency Calculation for 45 mg CBDA Puck

To manufacture 45 mg CBDA flower using B27 the calculation is as follows:

$$Potency of CBDA = 9.53 \%$$

$$\begin{matrix}Puck Weight for 45 mg CBDA = Defined Dose ( mg ) / Potency of CBDA \\ = 45 mg / 0.0953 \\ = 472 mg puck weight\end{matrix}$$

### Compression

The pucks were compressed at a puck weight of 472 mg ±5% to deliver 45 mg CBDA. The compression force used was 300-1500 PSI. At a compression force of 300-1500 PSI the pucks manufactured demonstrated acceptable integrity and hardness. The puck thickness of the 45 mg pucks was significantly larger than the 20 mg CBDA pucks. These pucks demonstrated good compressibility at lower compression force. The weight of each puck manufactured is provided in the table below.

45 mg CBDA Puck (Milled Flower 60 Mesh Sieve)

| | Puck Weight | | | | |
|---|---|---|---|---|---|
| | (mg) Target | | Dwell | Puck | Puck |
| | 472 mg ± 5% | Compression | Time | Diameter | Thickness |
| Description | (450-496 mg) | Force | (sec) | (mm) | (mm) |
| B27 | 473 | 300 | Less | 10.10 | 10.37 |
| (CBDA) | 448 | | than | 10.07 | 10.36 |
| | 454 | | 3 | 10.06 | 10.66 |
| | 495 | | | 9.87 | 10.96 |
| | 464 | | | 9.99 | 11.08 |
| | 471 | | | 9.99 | 10.59 |
| | 446 | | | 9.84 | 11.43 |
| | 455 | | | 9.87 | 9.09 |
| | 456 | | | 10.02 | 11.11 |
| | 450 | | | 10.13 | 10.20 |
| Average | 461 | | | 9.99 | 10.59 |

No. of pucks manufactured: 10

### Friability

Friability testing was performed on the 45 mg pucks and loss of 0.66% was obtained which is less than 1% which meets specifications provided by USP <1216>.

### Potency Data

B27 472 mg Puck Data

| | | mg per puck | | mg per puck |
|---|---|---|---|---|
| ID No. | % CBDA | CBDA | % CBD | CBD* |
| | 11.1 | 51.171 | 0.71 | 48.150067 |
| | 11.2 | 51.632 | 0.725 | 48.623514 |
| | 11.4 | 52.554 | 0.72 | 49.409058 |
| Average | 11.2333333 | 51.78566667 | 0.718333333 | 48.72754633 |
| SD | | 0.704189132 | | 0.63591008 |
| RSD | | 1.359814746 | | 1.305032015 |

The average potency for the theoretical B27 45 mg CBDA puck was 52 mg CBDA. The potency of CBD was calculated to be 49 mg CBD. The potency increased by approximately 10%.

### Conclusion

The puck prototypes manufactured were acceptable with respect to hardness. This study demonstrated that the B27 flower can be compressed at compression pressure of 300-1500 PSI for both the 20 mg and 45 mg puck, a compression force higher than 1500 PSI will lead to pucks with higher tensile strength. At this compression force, the friability and hardness of the pucks was not compromised. Puck are packaged in opaque bottles to protect from light

The potency data for both the 20 mg and 45 mg puck demonstrated that sieving the flower material through a 60 mesh sieve increased the potency by approximately 10%. In conclusion, the purity of the flower increased by implementing a dry sieving process.

The purpose of this study was to prepare pucks of B27 containing CBDA, sieved through a 60 mesh sieve and unsieved material to evaluate whether sieving can impact critical process parameters (CPPs) that can impact the critical quality attributes (CQAs) such as potency. The details of the studies are provided below. Pucks were manufactured with both the sieved and unsieved flower materials.

### Milling/Sieving of Flower

The sieved and milled material was used for the manufacturing of pucks. 66.5% was retained on 60 mesh sieve and 33.5% was sieved and retained on sieve pan.

| Theoretical | | | Amount | |
|---|---|---|---|---|
| Potency (%) | Lot No. | Sieve No. | Retain (g) | % Retain |
| 9.53% | 0143 | 60 | 6.31 | 66.5 |
| 9.53% | 0143 | Sieve Pan | 3.18 | 33.5 |

### Puck Manufacturing

20 mg CBDA (Milled B27 Flower Sieve Pan-Sieved through 60 Mesh)

| | Puck Weight | | | | Puck |
|---|---|---|---|---|---|
| | (mg) Target | Com- | Dwell | Puck | Thick- |
| | 210 mg ±5% | pression | Time | Diameter | ness |
| Description | (200-226 mg) | Force | (sec) | (mm) | (mm) |
| B27 | 211 | 1000 | Less | 9.76 | 3.28 |
| (CBDA) | 215 | | than | 9.78 | 3.19 |
| | 216 | | 3 | 9.82 | 3.10 |
| Average | 214 | | | 9.79 | 3.19 |

20 mg CBDA (Milled B27 Flower Retained on 60 Mesh Sieve)

| | Puck Weight | | | | |
|---|---|---|---|---|---|
| | (mg) Target | | Dwell | Puck | Puck |
| | 210 mg ±5% | Compression | Time | Diameter | Thickness |
| Description | (200-226 mg) | Force | (sec) | (mm) | (mm) |
| B27 | 207 | 1000 | Less | 9.68 | 2.85 |
| (CBDA) | 204 | | than | 9.68 | 2.90 |
| | 213 | | 3 | 9.63 | 2.81 |
| Average | 208 | | | 9.66 | 2.85 |

### Potency

Potency of Flower Material (Unmilled and Unsieved)

| % CBDA | % CBD | Total % CBD* |
|---|---|---|
| 6.5 | 0.4 | 6.9 |

CBDA and CBD Data for 210 mg Puck (Retained on 60 Mesh) [Hardeep Check]

| | | mg per puck | | mg per puck |
|---|---|---|---|---|
| | % CBDA | CBDA | % CBD | CBD* |
| | 3.9 | 8.3 | 0.280 | 7.8 |
| | 3.6 | 7.5 | 0.250 | 7.1 |
| | 3.9 | 8.2 | 0.275 | 7.8 |
| Average | 3.8 | 8.0 | 0.3 | 7.5 |
| SD | | 0.4 | | 0.4 |
| RSD | | 5.6 | | 5.6 |

CBDA and CBDA Data for 210 mg Milled Flower (Retained on 30 Mesh)

| % CBDA | % CBD | Total % CBD* |
|---|---|---|
| 3.8 | 0.7 | 4.1 |

CBDA and CBD Data for 210 mg Puck (Retained on Sieve Pan after Pass 60 Mesh)

| | | mg per puck | | mg per puck |
|---|---|---|---|---|
| ID No. | % CBDA | CBDA | % CBD | CBD* |
| | 11.1 | 23.3 | 0.710 | 21.9 |
| | 11.2 | 23.5 | 0.725 | 22.1 |
| | 11.4 | 23.9 | 0.720 | 22.5 |
| Average | 11.2 | 23.6 | 0.7 | 22.2 |
| SD | | 0.3 | | 0.3 |
| RSD | | 1.4 | | 1.3 |

CBDA and CBDA Data for 210 mg Milled Flower (Pass through 60 Mesh)

| % CBDA | % CBD | Total % CBD* |
|---|---|---|
| 11.8 | 0.710 | 11.1 |

### Dried and Milled Flower Potency

The potency of the flower material which was unsieved and unmilled was sent out for potency testing and the total %CBDA was 6.5% whereas the CoA stated the % CBDA to be 9.53%. There was drop in overall potency of plant by greater than 3%.

The milled flower that was retained on 60 mesh was tested for potency. The potency of this milled material was 3.8% CBDA and 4.1% CBD which is significantly less than CoA (certificate of analysis) potency of 9.53%.

The milled flower that passed through the 60 mesh sieve had potency of 11.8%CBDA and 11.1%CBD which is higher than the CoA potency.

### Puck Potency

Pucks manufactured with material retained on 60 mesh gave average potency 8.0 mg CBDA (7.5 mg CBD), which is below theoretical dose of 20 mg CBDA. However, based on the CofA potency this is a decrease in potency by 60%. Pucks manufactured with material passing through 60 mesh gave average potency of 23.6 mg CBDA (22.2 mg CBD) which is increase in potency by 15% for both CBDA and CBD

### Conclusion

Approximately 66.5% of B27 CBDA flower was retained on the 60 mesh sieve and 33.5% of B27 CBDA flower was sieved and retained on the sieve pan. The material retained on the 60 mesh sieve and material that passed through the 60 mesh sieve was compressed to evaluate whether it will impact potency.

The potency decreased by milling the flower material and passing through a 60 mesh sieve; the fraction of material retained in the 60 mesh sieve demonstrated a 60% decrease in potency. However, the flower material that was milled, passed through the 60 mesh sieve, and retained on the sieve pan showed increase in potency of puck by 15%.

The following examples provide defined dose cannabis pucks with standardized, consistent amounts of cannabinoids. In the following examples, the amount of cannabinoid is presented differently from the preceding examples. In the following examples, THC/A is the total of THCA plus THC, sometimes signified in the literature as THC_{TOT}; Similarly CBD/A is the total of CBDA plus CBD, and CBG/A is the total of CBGA plus CBG. These calculations are distinct from preceding examples where the total amount of, for example, THC was derived from converting THCA to THC and adding that derived amount to the amount of actual THC. The examples below report the combined mass of THCA and THC per cannabis puck. The same is reported for CBD/A and CBG/A

### Example 3

### Time Warp Flower Puck Containing Defined Dose of 20 mg THC/A

| R&D Lot | | Potency (%) | | Theoretical Fill | |
|---|---|---|---|---|---|
| No. | Formulation | (THC/A w/w) | Lot No. | Weight (mg) | Dose |
| RD-02P- | Time Warp | 9.40% | 0145 | 215 | 20 mg |
| 20 | (THC/A) | | | | THC/A |

Flower Preparation: Approximately 10 g of dried cannabis flower is milled gently to break up the largest pieces without generating significant fine particle content.

### Potency Calculation for 20 mg THC/A Puck

To manufacture 20 mg THC/A flower using Time Warp the calculation is as follows:

$$Potency of THC / A = 9.4 \% weight / weight of dried cannabis$$

$$\begin{matrix}Puck Weight for 20 mg THC / A = Defined Dose ( mg ) / Potency of THC / A \\ = 20 mg / 0.094 \\ = 215 mg puck weight\end{matrix}$$

Compression

The puck weight is chosen based on each puck being able to deliver 20 mg THC/A, based on the potency calculation. The pucks are compressed on the tablet press at a puck weight of 215 mg ±5% to deliver.

| | | Com- | Dwell | Puck | Puck | |
|---|---|---|---|---|---|---|
| Descrip- | Puck Weight | pression | Time | Diameter | Thickness | Volume |
| tion | (mg) | Force | (sec) | (mm) | (mm) | (mm3) |
| Time | 215 mg ± 5% | 1000 | Less | 9.8 | 3.28 | 247.4 |
| Warp | (205-226 mg) | | than 3 | | | |
| (THCA) | | | | | | |

The defined doses of principal cannabinoids in this puck are:

| Cannabinoid | Amount | |
|---|---|---|
| THCA | 18.4 | mg |
| THC | 1.6 | mg |
| Total THC/A | 20 | mg |
| CBDA | 0.16 | mg |
| CBD | 0.02 | mg |
| Total CBD/A | 0.18 | mg |
| CBNA | 0.13 | mg |

| | |
|---|---|
| CBN | Below detection |

### Example 4

### Time Warp Flower Puck Containing Defined Dose of 45 mg THC/A.

| | | Theoretical | | Theoretical | |
|---|---|---|---|---|---|
| R&D Lot | | Potency (%) | | Fill | |
| No. | Formulation | (THC/A w/w) | Lot No. | Weight (mg) | Dose |
| RD-02P- | Time Warp | 10.15% | 0145 | 479 | 45 mg |
| 45 | (THC/A) | | | | THC/A |

Flower Preparation: Approximately 10 g of dried cannabis flower is milled gently to break up the largest pieces without generating significant fine particle content.

### Potency Calculation for 45 mg THC/A Puck

To manufacture 45 mg THCA flower using Time Warp the calculation is as follows:

$$Potency of THC / A = 9.4 \%$$

$$\begin{matrix}Puck Weight for 49 mg THC / A = Defined Dose ( mg ) / Potency of THC / A \\ = 49 mg / 0.094 \\ = 479 mg puck weight\end{matrix}$$

Compression

| | | Com- | Dwell | Puck | Puck | |
|---|---|---|---|---|---|---|
| Descrip- | Puck Weight | pression | Time | Diameter | Thickness | Volume |
| tion | (mg) | Force | (sec) | (mm) | (mm) | (mm3) |
| Time | 479 mg ± 5% | 1000 | Less | 9.9 | 6.9 | 531.1 |
| Warp | (456-503 mg) | | than 3 | | | |

The defined doses of principal cannabinoids in this puck are:

| Cannabinoid | Amount | |
|---|---|---|
| THCA | 41.4 | mg |
| THC | 3.6 | mg |
| Total THC/A | 45 | mg |
| CBDA | 0.35 | mg |
| CBD | 0.05 | mg |
| Total CBD/A | 0.40 | mg |
| CBNA | 0.29 | mg |

| | |
|---|---|
| CBN | Below detection |

### Example 5

### B27 Flower Puck Containing Defined Dose of 20 mg CBD/A

(Note: CBD/A is the total of CBDA plus CBD, sometimes signified as CBD_{TOT})

| | | Theoretical | | Theoretical | |
|---|---|---|---|---|---|
| R&D Lot | | Potency (%) | | Fill | |
| No. | Formulation | (CBD/A w/w) | Lot No. | Weight (mg) | Dose |
| RD-01P- | B27 | 9.53% | 0143 | 210 | 20 mg |
| 20 | (CBD/A) | | | | CBD/A |

Flower Preparation: Approximately 10 g of dried cannabis flower is milled gently to break up the largest pieces without generating significant fine particle content.

### Potency Calculation for 20 mg CBD/A Puck

To manufacture 20 mg CBD/A flower using B27 flower the calculation is as follows:

$$Potency of CBD / A = 9.53 \%$$

$$\begin{matrix}Puck Weight for 20 mg CBD / A = Defined Dose ( mg ) / Potency of CBD / A \\ = 20 mg / 0.0953 \\ = 210 mg puck weight\end{matrix}$$

### Compression

| | | Com- | Dwell | Puck | Puck | |
|---|---|---|---|---|---|---|
| Descrip- | Puck Weight | pression | Time | Diameter | Thickness | Volume |
| tion | (mg) | Force | (sec) | (mm) | (mm) | (mm³) |
| B27 | 210 mg ± 5% | 150 | Less | 9.7 | 4.6 | 340 |
| (CBDA) | (200-226 mg) | | than 3 | | | |

The defined doses of principal cannabinoids in this puck are:

| Cannabinoid | Amount | |
|---|---|---|
| THCA | 0.92 | mg |
| THC | 0.08 | mg |
| Total THC/A | 1.0 | mg |
| CBDA | 17.5 | mg |
| CBD | 2.5 | mg |
| Total CBD/A | 20.0 | mg |

| | |
|---|---|
| CBNA | Below detection |
| CBN | Below detection |

### Example 6

### B27 Flower Puck Containing Defined Dose of 45 mg CBD/A

| | | Theoretical | | Theoretical | |
|---|---|---|---|---|---|
| R&D Lot | | Potency (%) | | Fill | |
| No. | Formulation | (CBD/A w/w) | Lot No. | Weight (mg) | Dose |
| RD-01P- | B27 | 9.53 | 0143 | 472 | 45 mg |
| 45 | (CBD/A) | | | | CBD/A |

Flower Preparation: Approximately 10 g of dried cannabis flower is milled gently to break up the largest pieces without generating significant fine particle content.

### Potency Calculation for 45 mg CBD/A Puck

To manufacture 45 mg CBD/A flower using B27 the calculation is as follows:

$$Potency of CBD / A = 9.53 \%$$

$$\begin{matrix}Puck Weight for 45 mg CBD / A = Defined Dose ( mg ) / Potency of CBD / A \\ = 45 mg / 0.0953 \\ = 472 mg puck weight\end{matrix}$$

| | | Com- | Dwell | Puck | Puck | |
|---|---|---|---|---|---|---|
| Descrip- | Puck Weight | pression | Time | Diameter | Thickness | Volume |
| tion | (mg) | Force | (sec) | (mm) | (mm) | (mm³) |
| B27 | 472 mg ± 5% | 150 | Less | 10.0 | 10.6 | 832.1 |
| (CBDA) | (450-496 mg) | | than 3 | | | |

The defined doses of principal cannabinoids in this puck are:

| Cannabinoid | Amount | |
|---|---|---|
| THCA | 2.10 | mg |
| THC | 0.15 | mg |
| Total THC/A | 2.25 | mg |
| CBDA | 39.4 | mg |
| CBD | 5.6 | mg |
| Total CBD/A | 45 | mg |

| | |
|---|---|
| CBNA | Below detection |
| CBN | Below detection |

### Example 7

### Enhanced Potency Time Warp Flower Puck Containing Defined Dose of 37.4 mg THC/A

This Example, and the following examples 8-10, describe cannabis pucks having enhanced potency. In these examples, the dried cannabis was prepared by milling and sieving the starting dried cannabis. Approximately 10 g of dried flower was placed into a mill and milled for 1-3 minutes to generate milled flower. The milled flower was then pushed through a 60 mesh sieve to a more fine powder. The powder has a light green colour to it. Only the fine powder of the first 2 grams was employed in the experiments and used for the manufacturing of pucks.

Also in Examples 5-8, in each case the density of flower material was measured by filling up a 10 mL graduated cylinder and weight was recorded and the mL occupied by the flower was also recorded. Afterwards, tapped density was recorded by tapping the flower in the graduated cylinder 100 times and the change in volume was recorded. The tapped density demonstrates compressibility of the flower. A higher tapped density entails good compressibility whereas lower density means poor compressibility.

Density of Flower=2.03 g/10 mL=0.203 g/mL

Tapped Density of Flower=2.03 g/8 mL=0.254 g/mL

| R&D | | Potency | Enhanced | | Fill | |
|---|---|---|---|---|---|---|
| Lot | Formu- | (%) (THC/A | Potency (%) | Lot | Weight | |
| No. | lation | w/w) | (THC/A w/w) | No. | (mg) | Dose |
| RD- | Time | 9.40% | 17.4% | 0145 | 215 | 37.4 mg |
| 02P-20 | Warp | | | | | THC/A |
| | (THCA) | | | | | |

### Compression

The puck weight was chosen based on each puck being able to deliver 37.4 mg THC/A, based on the potency calculation the pucks were compressed on the tablet press at a puck weight of 215mg ±5% to deliver. The compression force used initially was 300-1500 PSI. However, after two days the pucks began to fall apart. The compression force was adjusted to 1000 PSI which gave a more robust puck with the required hardness and puck integrity.

FIG. 3 provides an illustration of the described puck.

### 37.4 mg THC/A Pucks (Milled Time Warp Flower 60 Mesh Sieve)

| | Puck Weight | | | | |
|---|---|---|---|---|---|
| | (mg) Target | | Dwell | Puck | Puck |
| | 215 mg ±5% | Compression | Time | Diameter | Thickness |
| Description | (205-226 mg) | Force | (sec) | (mm) | (mm) |
| Time Warp | 215 | 1000 | Less | 9.77 | 3.62 |
| (THC/A) | 215 | | than | 9.77 | 3.57 |
| | 214 | | 3 | 9.83 | 3.33 |
| | 209 | | | 9.89 | 3.06 |
| | 206 | | | 9.78 | 3.09 |
| | 215 | | | 9.75 | 3.14 |
| | 203 | | | 9.82 | 3.19 |
| | 207 | | | 9.82 | 3.41 |
| | 207 | | | 9.76 | 3.11 |
| | 211 | | | 9.84 | 3.24 |
| | 215 | | | 9.77 | 3.17 |
| | 214 | | | 9.69 | 3.58 |
| | 218 | | | 9.83 | 3.06 |
| | 212 | | | 9.78 | 3.22 |
| | 208 | | | 9.87 | 3.62 |
| Average | 210 | | | 9.80 | 3.28 |

No. of pucks manufactured: 15

### Friability

Friability testing was performed on the 37.4 mg pucks and loss of 0.56% was obtained which is less than 1% according to the specifications provided by USP <1216>.

| | Weight | Weight | Friability |
|---|---|---|---|
| Description | Before (g) | After (g) | (%) |
| TIME | 1.07 | 1.064 | 0.56% |
| Warp | | | |
| (THC/A) | | | |

Packaging

The pucks were packaged into white opaque bottles with child resistant caps and stored at 4° C.

The potency of the pucks was evaluated. The defined doses of principal cannabinoids in this puck are:

| Cannabinoid | Amount | |
|---|---|---|
| THCA | 35.0 | mg |
| THC | 2.4 | mg |
| Total THC/A | 37.4 | mg |
| CBDA | 0.30 | mg |
| CBD | 0.04 | mg |
| Total CBD/A | 0.34 | mg |
| CBNA | 0.25 | mg |

| | |
|---|---|
| CBN | Below detection |

### Example 8

### Enhanced Potency Time Warp Flower Puck Containing Defined Dose of 77.1 mg THC/A

| | | | Enhanced | | | |
|---|---|---|---|---|---|---|
| R&D | | Theoretical | Potency | | Theoretical | |
| Lot | Formu- | Potency | (%) (THC/ | Lot | Fill Weight | |
| No. | lation | (%) | A w/w) | No. | (mg) | Dose |
| RD- | Time | 9.40% | 16.1% | 0145 | 479 | 77.1 mg |
| 02P-45 | Warp | | | | | THC/A |
| | (THC/A) | | | | | |

### Compression

To deliver 77.1 mg THC/A it was found that we needed to compress pucks with a weight of 479 mg ±5% to deliver 77.1 mg THCA. A compression force of 1000 PSI gave a robust puck with the required hardness. The puck diameter and puck thickness of these pucks compared to 34 mg THCA pucks is significantly larger. FIG. 4A provides an illustration of the described puck.

### 77.1 mg THCA Puck (Milled Flower, 60 Mesh Sieve)

| | Puck Weight | | | | |
|---|---|---|---|---|---|
| | (mg) Target | | Dwell | Puck | Puck |
| | 479 mg ±5% | Compression | Time | Diameter | Thickness |
| Description | (456-503 mg) | Force | (sec) | (mm) | (mm) |
| Time Warp | 459 | 1000 | Less | 9.9 | 6.72 |
| (THCA) | 462 | | than | 9.82 | 6.82 |
| | 461 | | 3 | 9.88 | 7 |
| | 482 | | | 9.82 | 6.96 |
| | 462 | | | 9.8 | 6.83 |
| | 471 | | | 9.77 | 6.82 |
| | 471 | | | 9.87 | 6.99 |
| | 473 | | | 9.85 | 6.94 |
| | 481 | | | 9.88 | 6.94 |
| | 480 | | | 9.83 | 6.89 |
| Average | 471 | | | 9.84 | 6.91 |

No. of pucks manufactured: 10

### Friability

Friability testing was performed on the 77.1 mg pucks and loss of 0.17% was obtained which is less than 1% according to the specifications provided by USP <1216>.

| | Weight | Weight | Friability |
|---|---|---|---|
| Description | Before (g) | After (g) | (%) |
| Time Warp | 2.354 | 2.35 | 0.17% |
| (THCA) | | | |

### Packaging

The pucks were packaged into white opaque bottles with child resistant caps and stored at 4° C.

The pucks were to be shipped to an outside lab for potency testing.

The defined doses of principal cannabinoids in this puck are:

| Cannabinoid | Amount | |
|---|---|---|
| THCA | 71.0 | mg |
| THC | 6.1 | mg |
| Total THC/A | 77.1 | mg |
| CBDA | 0.38 | mg |
| CBD | Below detection | |
| Total CBD/A | 0.38 | mg |
| CBNA | 0.55 | mg |

| | |
|---|---|
| CBN | Below detection |

The pucks manufactured were acceptable with respect to hardness. Examples 5 and 6 demonstrated that the Time Warp flower can be compressed at compression pressure of 1000 PSI, a compression force less than 1000 PSI will lead to pucks with lower tensile strength and lead to other puck defects. It is recommended to manufacture pucks at compression pressures between 1000-1500 psi for the Time Warp flower. At this compression force the friability and hardness of the pucks was not compromised. Pucks are packaged into opaque bottles to protect from light.

### Example 9

### Enhanced Potency B27 Flower Puck Containing Defined Dose of 25.4 mg CBD/A.

| | | Theoretical | Enhanced | | | |
|---|---|---|---|---|---|---|
| | | Potency | Potency | | | |
| R&D | | (%) | (%) | | Fill | |
| Lot | Formu- | (CBD/A | (CBD/A | Lot | Weight | |
| No. | lation | w/w) | w/w) | No. | (mg) | Dose |
| RD- | B27 | 9.53% | 12.1% | 0143 | 210 | 25.4 mg |
| 01P-20 | (CBDA) | | | | | CBDA |

### Compression

The optimum weight of the puck was determined based on delivering 25.4 mg CBDA which gave a puck weight of 210 mg. The pucks were compressed at a puck weight of 210 mg ±5% to deliver 25.4 mg CBDA. The weight of each puck manufactured is provided in the table below. FIG. 4A provides an illustration of the described puck.

25.4 mg CBDA (Milled B27 Flower 60 Mesh Sieve)

| | Puck Weight | | | | |
|---|---|---|---|---|---|
| | (mg) Target | | Dwell | Puck | Puck |
| | 210 mg ±5% | Compression | Time | Diameter | Thickness |
| Description | (200-226 mg) | Force | (sec) | (mm) | (mm) |
| B27 | 210 | 150 | Less | 9.71 | 4.58 |
| (CBDA) | 203 | | than | 9.90 | 4.62 |
| | 207 | | 3 | 9.90 | 4.41 |
| | 204 | | | 9.87 | 5.09 |
| | 207 | | | 9.90 | 4.86 |
| | 202 | | | 9.95 | 4.73 |
| | 207 | | | 9.81 | 4.17 |
| | 210 | | | 9.89 | 4.57 |
| | 202 | | | 9.88 | 4.60 |
| | 206 | | | 9.58 | 4.41 |
| | 211 | | | 9.82 | 4.62 |
| | 210 | | | 9.77 | 4.26 |
| | 202 | | | 9.73 | 3.84 |
| | 212 | | | 9.87 | 4.44 |
| | 205 | | | 9.85 | 4.76 |
| Average | 207 | | | 9.83 | 4.53 |

No. of pucks manufactured: 15

### Friability

Friability testing was performed on the 25.4 mg pucks and loss of 0.9% was obtained which is less than 1% and meets specifications provided by USP <1216> Friability testing was performed by weighing 5 pucks and recording the weight and putting them into a bag and rotating 100 times to mimic a friability tester and weighing all 5 pucks after the friability test.

| | Weight | Weight | Friability |
|---|---|---|---|
| Description | Before (g) | After (g) | (%) |
| B27 | 1.010 | 1.001 | 0.9% |
| (CBDA) | | | |

### Packaging

The pucks were packaged into white opaque bottles with child resistant caps and stored at 4° C. The defined doses of principal cannabinoids in this puck are:

| Cannabinoid | Amount | |
|---|---|---|
| THCA | 1.1 | mg |
| THC | 0.2 | mg |
| Total THC/A | 1.3 | mg |
| CBDA | 23.9 | mg |
| CBD | 1.5 | mg |
| Total CBD/A | 25.4 | mg |

| | |
|---|---|
| CBNA | Below detection |
| CBN | Below detection |

### Example 10

### Enhanced Potency B27 Flower Puck Containing Defined Dose of 56.4 mg CBD/A.

Goal: To evaluate compression of puck prototypes at puck weight of 472 mg and to determine optimal compression force required to manufacture pucks with acceptable integrity.

| | | Theoretical | Enhanced | | | |
|---|---|---|---|---|---|---|
| | | Potency | Potency | | Theoretical | |
| R&D | | (%) | (%) | | Fill | |
| Lot | Formu- | (CBD/A | (CBD/A | Lot | Weight | |
| No. | lation | w/w) | w/w) | No. | (mg) | Dose |
| RD- | B27 | 9.53 | 11.9% | 0143 | 472 | 56.4 mg |
| 01P-45 | (CBDA) | | | | | CBD/A |

### Compression

The pucks were compressed at a puck weight of 472 mg ±5% to deliver 56.4 mg CBD/A. The compression force used was 300-1500 PSI. At a compression force of 300-1500 PSI the pucks manufactured demonstrated acceptable integrity and hardness. The puck diameter and thickness of the 472 mg pucks was significantly larger than the 34 mg CBD/A pucks. These pucks demonstrated good compressibility at lower compression force. The weight of each puck manufactured is provided in the table below. FIG. 4A and FIG. 4B provides an illustration of the described puck.

56.4 mg CBD/A Puck (Milled Flower 60 Mesh Sieve)

| | Puck Weight | | | | |
|---|---|---|---|---|---|
| | (mg) Target | | Dwell | Puck | Puck |
| | 472 mg ±5% | Compression | Time | Diameter | Thickness |
| Description | (450-496 mg) | Force | (sec) | (mm) | (mm) |
| B27 | 473 | 150 | Less | 10.10 | 10.37 |
| (CBDA) | 448 | | than | 10.07 | 10.36 |
| | 454 | | 3 | 10.06 | 10.66 |
| | 495 | | | 9.87 | 10.96 |
| | 464 | | | 9.99 | 11.08 |
| | 471 | | | 9.99 | 10.59 |
| | 446 | | | 9.84 | 11.43 |
| | 455 | | | 9.87 | 9.09 |
| | 456 | | | 10.02 | 11.11 |
| | 450 | | | 10.13 | 10.20 |
| Average | 461 | | | 9.99 | 10.59 |

No. of pucks manufactured: 10

### Friability

Friability testing was performed on the 56.4 mg pucks and loss of 0.66% was obtained which is less than 1% which meets specifications provided by USP <1216> Friability testing was performed by weighing 5 pucks and recording the weight and putting them into a bag and rotating 100 times to mimic a friability tester and weighing all 5 pucks after the friability test.

| | Weight | Weight | Friability |
|---|---|---|---|
| Description | Before (g) | After (g) | (%) |
| B27 | 2.266 | 2.251 | 0.66 |
| (CBDA) | | | |

### Packaging

The pucks were packaged into white opaque bottles with child resistant caps and stored at 4° C. The defined doses of principal cannabinoids in this puck are:

| Cannabinoid | Amount | |
|---|---|---|
| THCA | 2.5 | mg |
| THC | 0.4 | mg |
| Total THC/A | 2.9 | mg |
| CBDA | 52.9 | mg |
| CBD | 3.5 | mg |
| Total CBD/A | 56.4 | mg |

| | |
|---|---|
| CBNA | Below detection |
| CBN | Below detection |

The puck prototypes manufactured were acceptable with respect to hardness. This study demonstrated that the B27 flower can be compressed at compression pressure of 300-1500 PSI; a compression force higher than 1500 PSI will lead to pucks with higher tensile strength and could impact vaporizing. At this compression force the friability and hardness of the pucks was also not compromised and pucks should be packaged into opaque bottles to protect from light.

### Example 11

### Purple X Chemo Flower Puck Containing Defined Dose of 45 mg THC/A and 13.4 mg CBG/A

| | | | | Dose | Dose |
|---|---|---|---|---|---|
| | | | | Calculated by | Calculated by |
| | Theoretical | Theoretical | | composition | conversion to |
| R&D Lot | Potency (%) | Potency (%) | Theoretical Fill | (total mass of | decarboxylated |
| No. | (THC/A w/w) | (CBG/A w/w) | Weight (mg) | cannabinoid) | amounts |
| BB47-S5 | 14.88 | 4.44 | 302 | 45 mg THC/A | 39.7 mg THC* |
| | | | | 13.4 mg | 11.9 mg CBG* |
| | | | | CBG/A | |

Flower Preparation: Approximately 10 g of dried cannabis flower is milled gently to break up the largest pieces without generating significant fine particle content.

### Potency Calculation for 45 mg THC/A and 13.4 mg CBG/A Puck

To manufacture a 45 mg THC/A flower using Purple X Chemo the calculation is as follows:

$$Potency of THC / A = 14.88 \%$$

$$\begin{matrix}Puck Weight for 45 mg THC / A = Defined Dose ( mg ) / Potency of THC / A \\ = 45 mg / 0.1488 \\ = 302 mg puck weight\end{matrix}$$

The puck is formed using 302 mg of prepared flower under compression (1000 PSI, Less than 3 seconds, room temperature) in the form of one of the shapes of the invention suitable for a vaporizer device. The defined dose of principal cannabinoids and principal terpenes in this puck are:

| Substance | Amount | | |
|---|---|---|---|
| THCA | 43.8 | mg | |
| THC | 1.2 | mg | |
| Total THC/A | 45 | mg | |
| CBDA | 0.02 | mg | |
| CBD | Not detected | | |
| Total CBD/A | 0.02 | mg | |
| CBGA | 12.8 | mg | |
| CBG | 0.6 | mg | |
| Total CBG/A | 13.4 | mg | |
| Total | | | 58.4 mg |
| Cannabinoid | | | |
| a-Pinene | 0.127 | mg | |
| B-Pinene | 0.103 | mg | |
| Myrcene | 0.085 | mg | |
| D-Limonene | 0.175 | mg | |
| Ocimene | 0.649 | mg | |
| Fenchone | 0.021 | mg | |
| Fenchol | 0.042 | mg | |
| Caryophyllene | 0.073 | mg | |
| Guaiol | 0.042 | mg | |
| a-Bisabolol | 0.003 | mg | |
| Total Terpene | | | 1.32 mg |

### Example 12

### Use of Pucks of the Invention with DaVinci IQ Vaporizer Device

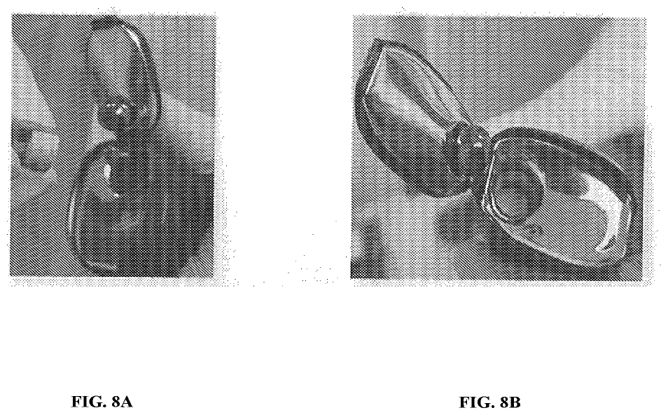

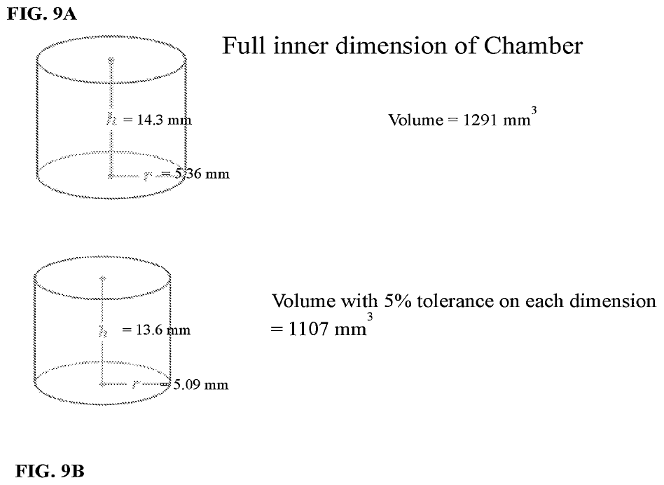

The compressed pucks of Example 6 and Example 8 above were designed to fit into the main flower chamber of the DAVINCI IQ™ VAPORIZER from Organicix (Las Vegas, Nev.). The DaVinci Vaporizer is a commercial handheld vaporizer device that offers safe and efficient administration of medicinal cannabis (Lanz C, Mattsson J, Soydaner U, Brenneisen R (2016) Medicinal Cannabis: In Vitro Validation of Vaporizers for the Smoke-Free Inhalation of Cannabis. PLOS ONE 11(1): e0147286. https://doi.org/10.1371/journal.pone.0147286). FIG. 8A and FIG. 8B illustrate the insertion of the Cannabis puck into the vaporizer bowl. FIG. 9A and FIG. 9B show the precise measurements of the bowl and the dimensions of a Cannabis puck of the invention, respectively.

The composition and methods described herein are illustrative and not limiting on the claims of the invention more particularly set out below.

1-34. (canceled)

35. A defined dose Cannabis composition comprising a defined dose of:
(a) 0.1-100 mg tetrahydrocannabinolic acid (THCA),
(b) 0.1-100 mg tetrahydrocannabinol (THC),
(c) 0.1-100 mg cannabidiolic acid (CBDA), and/or
(d) 0.1-100 mg cannabidiol (CBD) in a friable puck comprising compressed ground Cannabis material.

36. The composition of claim 35, wherein the Cannabis material includes material derived from one or more Cannabis plants from a species selected from the group consisting of Cannabis sativa, Cannabis indica, Cannabis ruderalis, and any hybrid thereof

37. The composition of claim 35, wherein the Cannabis species is Cannabis indica.

38. The composition of claim 35, wherein the Cannabis material includes material prepared from Cannabis inflorescence.

39. The composition of claim 35, further comprising additives selected from the group consisting of terpenes, terpenoids, puck stabilizers, humectants, vaporization aids, fillers flavours, and any combination thereof.

40. The composition of claim 35, wherein the amount of THC is less than a psychotropic dose.

41. The composition of claim 35, wherein the amount of THCA is less than 1.0 mg.

42. The composition of claim 35, comprising
THCA in an amount between 5-165 mg,
THC in an amount less than 1.0 mg; and
CBDA in an amount between 0.1-70 mg, and having total mass 100-500 mg.

43. The composition of claim 35, comprising
THCA in an amount less than 5.0 mg,
THC in an amount between 1-5 mg or between 5-135 mg; and
CBD in an amount between 0.1-70 mg, and having total mass 100-500 mg.

44. The composition of claim 35, comprising
THCA in an amount less than 1.0 mg
THC in an amount less than 1.0 mg; and
CBD in an amount between 5-90 mg, and having total mass 100-500 mg.

45. The composition of claim 44 wherein the Cannabis material is derived from one or more Cannabis plant varieties selected from the group consisting of Charlottes' Web and one or more other high CBD, low THCA Cannabis plant varieties.

46. The composition of claim 35, further comprising a plurality of air channels of diameter not greater than 0.5 mm and disposed within the friable puck, such that no portion of the puck is greater than about 0.5, 1, 1.5, 2, 2.5, or 3 mm from an air surface.

47. The composition of claim 35, having a high-gloss surface.

48. The composition of claim 35, further comprising a blister package, impermeable to gas exchange, for containing the friable puck.

49. The composition of claim 48, wherein the blister package is configured to contain the friable puck in a sealed, inert gas atmosphere.

50. The composition of claim 35, further comprising a re-sealable package for containing one or more of the friable pucks, wherein in a sealed configuration the package provides an environment for containing the pucks that is impermeable to gas exchange.

51. The composition of claim 35, wherein the composition comprises a signifier for providing visual information on one or more defined dose(s) of one or more selected cannabinoids in the composition.

52. The composition of claim 48, wherein the package comprises a signifier for providing visual information on the defined dose(s) of one or more selected cannabinoids in the composition.

53. The composition of claim 35, wherein said puck has a total mass of about 45 mg or from about 0.05 g to about 1.0 g.

54. The composition of claim 35, wherein the composition possesses a degree of friability such that no more than, or exactly, 1% or 0.66% loss results after a friability test as per <USP 1216>.

55. The composition of claim 35, wherein the puck possesses a degree of friability that meets specifications provided by USP <1216>.

56. The composition of claim 35, wherein the compressed ground Cannabis material is milled or ground before being incorporated into the composition.

57. The composition of claim 56, wherein material is milled or ground and then passed through a mesh or a sieve before being incorporated into the composition.

58. The composition of claim 57, wherein the mesh or sieve has a mesh size of 30, 60, or 120.

59. The composition of claim 57, wherein the mesh or sieve has an average opening size of about 0.595 mm, about 0.250 mm, or about 0.125 mm.

60. The composition of claim 57, wherein the mesh or sieve has a mesh size of 60.

61. The composition of claim 57, wherein the mesh or sieve has an average opening size of about 0.250 mm.

62. The composition of claim 35, wherein the composition is formed by compressing its components into a predetermined shape.

63. The composition of claim 62, wherein the shape is a puck shape.

64. The composition of claim 62, wherein the shape is predetermined to be received by a Cannabis vaporizer.

65-87. (canceled)
